SEQUENCE LISTING (12 sequences)

SEQ ID NO: 1 - protein, 850 aa, Homo sapiens
MAAQRNRSKESKDCSGLVLLCLFFGIPWEAGARQISYSIPEELEKGSFVGNISKDLGLAPRELAERGVRIVSRGRTQLFSLNPRSGSLITAGRIDREELCAQSARCVVSFNILVEDRVKLFGIEIEVTDINDNAPKFQAENLDVKINENVAAGMRFPLPEAIDPDVGVNSLQSYQLSPNKHFSLRVQSRANGVKYPELVLEHSLDREEEAIHHLVLTASDGGDPLRSGTVLVSVTVFDANDNAPVFTLPEYRVSVPENLPVGTQLLTVTATDRDEGANGEVTYSFRKLPDTQLLKFQLNKYTGEIKISENLDYEETGFYEIEIQAEDGGAYLATAKVLITVEDVNDNSPELTITSLFSPVTEDSPLGTVVALLNVHDLDSEQNGQVTCSILAYLPFKLEKSIDSYYRLVIHRALDREQVSSYNITVTATDGGSPPLSTEAHFMLQVADINDNPPTFSQVSYFTYIPENNARGASIFSVTALDPDSKENAQIIYSLAEDTIQGVPLSSYISINSDTGVLYALRSFDYEQFHELQMQVTASDSGDPPLSSNVSLSLFVLDQNDNAPEILYPALPTDGSTGVELAPRSAEPGYLVTKVVAVDRDSGQNAWLSYRLLKASEPGLFAVGEHTGEVRTARALLDRDALKQSLVVAVQDHGQPPLSATVTLTVAVADSIPQVLADLGSFESPANSETSDLTLYLVVAVAAVSCVFLAFVIVLLAHRLRRWHKSRLLQASGGGLTGVSGSHFVGVDGVRAFLQTYSHEVSLTADSRKSHLIFPQPNYADTLISQESCEKNDPLSLLDDSKFPIEDTPLVPVSFIFIFTFVKKKKIGFYFEVCGMMVESVNAKTLMSRI

SEQ ID NO: 2 - DNA, 2553 bp, Homo sapiens
  source 1..2553: mol_type = genomic DNA
atggccgctcaaaggaatcgctcaaaggaatcaaaggattgcagcgggctggtcctgctctgccttttcttcgggattccatgggaggctggagcccggcagatctcctactcaattcctgaggaattagagaaaggctctttcgtgggcaacatctccaaggacttgggtctggcgccccgggagctggcggagcgcggagtccgcatagtctccagaggtaggacgcagcttttctctctgaacccgcgcagcggcagcttgatcaccgcgggcaggatagaccgggaggagctctgcgctcagagcgcgcggtgcgtggtgagttttaatatccttgtggaagacagggtgaaactttttgggatagaaatagaagtaactgatatcaatgacaatgctccaaaattccaagcagaaaatctagacgtaaaaattaatgaaaatgtcgctgcgggaatgcgttttcctctcccggaagctattgatccggatgtgggcgtgaactccctgcagagctatcagctcagccccaataagcacttctccctaagagttcagagccgtgccaatggcgtcaagtacccggagctggtactggagcactccctagatcgcgaggaagaggccattcaccacctggtcctcaccgcctccgacgggggtgaccctctccgatctggcactgtccttgtcagtgtgactgtcttcgatgcaaatgacaacgcgccggtcttcaccttgccagaataccgagtgagtgttcctgagaatttgcctgtgggcactcagctgctgacagtcacagccaccgacagggacgaaggtgccaatggagaagtgacatattcattccgaaaattacctgacacgcaattgttgaagttccaactaaacaaatatactggagaaataaaaatatcagaaaatctagattatgaagaaaccggtttctatgaaatagaaatacaagcagaagatggaggagcatatcttgcaactgcaaaagtgttgattacagtagaagatgtaaatgacaacagtccagagctgaccatcacgtctctatttagtccagtgactgaagattcacctctgggaacagtcgtagcccttttaaatgtgcatgatttagactctgagcagaatggacaggtaacctgttccattttggcgtatctaccatttaaattagaaaagtccattgacagttattacagattggtgatacacagagcccttgacagggaacaggtatcctcttacaatatcacagtgacagccacagatgggggaagtcctcctctatcaacggaagctcactttatgctacaagtggcagatatcaatgacaacccacctaccttctctcaagtctcctactttacctatatcccagagaacaacgccaggggtgcctccatcttctcagtgacagcgctggacccggacagcaaagagaatgcccagattatttactccctggctgaagacaccatccagggggtacctctgtcctcatacatatccatcaactcagacactggcgtcctgtatgcactcagatccttcgactatgagcagtttcatgagctacagatgcaggtgacagccagcgacagcggggatcctccactcagcagcaacgtgtcgttgagcctgtttgtgctggaccagaacgacaatgcgcccgagatcctgtaccccgccctccccacagacggttccacaggcgtggagctggcgccccgctccgcagagcccggctacctggtgaccaaggtggtggcggtggacagagactccggccagaacgcctggctgtcctaccgtctgctcaaggccagcgagccgggactcttcgcggtgggggagcacacgggcgaggtgcgcacggcgcgagccctgctggacagagacgcgctcaagcaaagcctcgtagtggccgtccaggaccacggccagccccctctctccgccactgtcacgctcaccgtggccgtggccgacagcatcccccaagtcctggcggacctcggcagcttcgagtctccagctaactctgaaacctcagacctcactctgtacctggtggtagcggtggccgcggtctcctgcgtcttcctggccttcgtcatcgtgctgctggcgcacaggctgcggcgctggcacaagtcacgcctgctgcaggcttcaggaggcggcttgacaggtgtgtccggctcgcactttgtgggcgtggacggggttcgggctttcctgcagacctattcccacgaggtctctctcaccgcggactcgcgaaagagtcacctgatcttcccccagcccaattatgcggacacgctcatcagccaggagagctgtgagaaaaacgatcctttgtctttgttagatgattcgaagtttcctatagaggataccccattggttccagtgagttttattttcatttttacttttgttaaaaaaaaaaagattggtttttactttgaagtttgcggcatgatggtggaaagtgtaaatgctaaaacactgatgagtagaatttga

SEQ ID NO: 3 - protein, 521 aa, Homo sapiens
MKSQGQHWYSSSDKNCKVSFREKLLIIDSNLGVQDVENLKFLCIGLVPNKKLEKSSSASDVFEHLLAEDLLSEEDPFFLAELLYIIRQKKLLQHLNCTKEEVERLLPTRQRVSLFRNLLYELSEGIDSENLKDMIFLLKDSLPKTEMTSLSFLAFLEKQGKIDEDNLTCLEDLCKTVVPKLLRNIEKYKREKAIQIVTPPVDKEAESYQGEEELVSQTDVKTFLEALPQESWQNKHAGSNGNRATNGAPSLVSRGMQGASANTLNSETSTKRAAVYRMNRNHRGLCVIVNNHSFTSLKDRQGTHKDAEILSHVFQWLGFTVHIHNNVTKVEMEMVLQKQKCNPAHADGDCFVFCILTHGRFGAVYSSDEALIPIREIMSHFTALQCPRLAEKPKLFFIQACQGEEIQPSVSIEADALNPEQAPTSLQDSIPAEADFLLGLATVPGYVSFRHVEEGSWYIQSLCNHLKKLVPRMLKFLEKTMEIRGRKRTVWGAKQISATSLPTAISAQTPRPPMRRWSSVS

SEQ ID NO: 4 - DNA, 2307 bp, Homo sapiens
  source 1..2307: mol_type = genomic DNA
aaggaaatggagatccaaaggaaagcctgaagcactttgtggcttccacgggttcgtttctaggaagcttttgctttacctggggaaaccccaagctctacagtgagaaagttgtaaattagcctttaggagacgctgtttgtcaaagttatcttaaccctggaaaaggggagggaaaaaggcagataagcagtgatgacatcagcaggaaactagtatcaggctacccagtcctgaagattcagttttcacttaaacaaccagcaagtcttgaagtctcttcccaagcaaatgggagcttctttggaccttggagcacacagaggattctactttctttaaaactttgttttcaggcaatttccctgagaaccgtttacttccagaagattggtggagcttgatctgaaggctggccatgaaatctcaaggtcaacattggtattccagttcagataaaaactgtaaagtgagctttcgtgagaagcttctgattattgattcaaacctgggggtccaagatgtggagaacctcaagtttctctgcataggattggtccccaacaagaagctggagaagtccagctcagcctcagatgtttttgaacatctcttggcagaggatctgctgagtgaggaagaccctttcttcctggcagaactcctctatatcatacggcagaagaagctgctgcagcacctcaactgtaccaaagaggaagtggagcgactgctgcccacccgacaaagggtttctctgtttagaaacctgctctacgaactgtcagaaggcattgactcagagaacttaaaggacatgatcttccttctgaaagactcgcttcccaaaactgaaatgacctccctaagtttcctggcatttctagagaaacaaggtaaaatagatgaagataatctgacatgcctggaggacctctgcaaaacagttgtacctaaacttttgagaaacatagagaaatacaaaagagagaaagctatccagatagtgacacctcctgtagacaaggaagccgagtcgtatcaaggagaggaagaactagtttcccaaacagatgttaagacattcttggaagccttaccgcaggagtcctggcaaaataagcatgcaggtagtaatggtaacagagccacaaatggtgcaccaagcctggtctccagggggatgcaaggagcatctgctaacactctaaactctgaaaccagcacaaagagggcagctgtgtacaggatgaatcggaaccacagaggcctctgtgtcattgtcaacaaccacagctttacctccctgaaggacagacaaggaacccataaagatgctgagatcctgagtcatgtgttccagtggcttgggttcacagtgcatatacacaataatgtgacgaaagtggaaatggagatggtcctgcagaagcagaagtgcaatccagcccatgccgacggggactgcttcgtgttctgtattctgacccatgggagatttggagctgtctactcttcggatgaggccctcattcccattcgggagatcatgtctcacttcacagccctgcagtgccctagactggctgaaaaacctaaactctttttcatccaggcctgccaaggtgaagagatacagccttccgtatccatcgaagcagatgctctgaaccctgagcaggcacccacttccctgcaggacagtattcctgccgaggctgacttcctacttggtctggccactgtcccaggctatgtatcctttcggcatgtggaggaaggcagctggtatattcagtctctgtgtaatcatctgaagaaattggtcccaaggatgctgaaatttctggaaaagacaatggaaatcaggggcaggaagagaacagtgtggggtgctaaacagatctcagcaacctccctgcccacggccatctctgcgcagacacctcgaccccccatgcgcaggtggagcagcgtttcctagttctttccagaggcttccttctgcctgccttccagccacatcgcctgagattgacaacgccctacagcaagacggaaacctccctttacagcaccaccttgcgattctgcagccacaaagttgagacttctgaacgtggcactcttctgttcccttactgtttcacgtgtacctgtgtcatctttcttgtttcatcgtaaacatacttctaaaattcccattttctttatttagaaatagaactacaagcggatggttaaacaatttaaacaaatggtccatggggaaaagtgaatttcacactgtcccccaaactttcagtg

SEQ ID NO: 5 - DNA, 20 bp, synthetic construct
  source 1..20: mol_type = other DNA
aagagtcacctgatcttccc

SEQ ID NO: 6 - DNA, 25 bp, synthetic construct
  source 1..25: mol_type = other DNA
acactggagtaaaaaccaatctttt

SEQ ID NO: 7 - DNA, 27 bp, synthetic construct
  source 1..27: mol_type = other DNA
acactcgagctgtgagaaaaaagatcc

SEQ ID NO: 8 - DNA, 28 bp, synthetic construct
  source 1..28: mol_type = other DNA
acatctagatttgggctcaagcacaacg

SEQ ID NO: 9 - DNA, 19 bp, synthetic construct
  source 1..19: mol_type = other DNA
aggccctcattcccattcg

SEQ ID NO: 10 - DNA, 19 bp, synthetic construct
  source 1..19: mol_type = other DNA
tataccagctgccttcctc

SEQ ID NO: 11 - DNA, 20 bp, synthetic construct
  source 1..20: mol_type = other DNA
tgccctgggaatccgcagct

SEQ ID NO: 12 - DNA, 20 bp, synthetic construct
  source 1..20: mol_type = other DNA
taagtaaaatcagaactcac

(19) United States
(12) Patent Application Publication
(10) Pub. No.: US 2024/0026455 A1
(43) Pub. Date: Jan. 25, 2024

(54) GENETIC VARIATIONS ASSOCIATED WITH BENIGN PAROXYSMAL POSITIONAL VERTIGO

(71) Applicant: Father Flanagan's Boys' Home Doing Business as Boys Town National Research Hospital, Omaha, NE (US)

(72) Inventor: Yesha (Yunxia) W. Lundberg, Omaha, NE (US)

(21) Appl. No.: 18/356,417

(22) Filed: Jul. 21, 2023

Related U.S. Application Data
(63) Continuation of application No. PCT/US22/13524, filed on Jan. 24, 2022.
(60) Provisional application No. 63/140,598, filed on Jan. 22, 2021.

(51) Int. Cl. C12Q 1/6883; C07K 16/18

(52) U.S. Cl. CPC C12Q 1/6883; C07K 16/18; C12Q 2600/156; C07K 2317/32

(57) ABSTRACT

The present disclosure relates generally to methods of diagnosing Benign Paroxysmal Positional Vertigo (BPPV) in a subject. In particular, the present disclosure relates to methods of genotyping a subject in order to determine the presence or absence of a genetic variation or variations indicative of BPPV. In other embodiments, an inhibitor is administered to a subject, wherein the inhibitor prevents aggregation of proteins in neural cells such as the vestibular ganglia. The disclosure further includes methods of identifying an agent that inhibits the aggregation of proteins in neural cells. Methods of monitoring protein aggregation in neural cells are also provided.

### CROSS-REFERENCE TO RELATED APPLICATIONS

This application is a Continuation Application of PCT/US2022/013526, filed on Jan. 24, 2022, which claims priority under 35 U.S.C. § 119 to provisional patent application U.S. Ser. No. 63/140,598, filed Jan. 22, 2021 all of which are herein incorporated by reference in their entirety, including without limitation, the specification, claims, and abstract, as well as any figures, tables, appendices, or drawings thereof.

### GOVERNMENT FUNDING

This invention was made with government support under Grant No. DC014748, awarded by the National Institutes of Health. The Government has certain rights in the invention.

### REFERENCE TO SEQUENCE LISTING SUBMITTED ELECTRONICALLY

The instant application contains a Sequence Listing which has been submitted electronically in XML, format and is herein incorporated by reference in its entirety. Said XML, copy, created on Jul. 20, 2023, is named “P13505US01_SequenceListing.xml” and is 17,925 bytes in size.

### BACKGROUND

Vertigo and balance deficits involving the vestibule of the inner ear are common, disruptive to daily living, and can be debilitating especially in the elderly, yet the molecular etiology of these conditions in humans is completely unknown. With a lifetime prevalence of 10%, Benign Paroxysmal Positional Vertigo (BPPV) is the most common cause of vertigo in humans (Von Brevern et al., 2015, J Vestib Res; 25:105-117). Clinically, BPPV is believed to be caused by otoconia dislocation from the utricle (which detects linear head motion) to the semicircular canals (which sense rotational movement), although visualization of such events has not been possible. Otoconia are extracellular bio-crystals overlaying the sensory epithelium of the utricle and saccule in the inner ear and are composed of proteins and calcium carbonate. The crystals provide inertia mass that yield shearing force on the stereocilia of the sensory hair cells during head movement.

Triggered by rapid changes of head position relative to gravity, BPPV episodes can be intense, and often cause nausea and vomiting. Therefore, the word “benign” in the term BPPV is only in the sense that the condition is not life-threatening; the vertigo itself is not benign at all. In fact, BPPV can be debilitating and disruptive to daily living and is much more incapacitating and persistent in the elderly (Handa et al., 2005, Braz J Otorhinolaryngol; 71:776-782; and Lopez-Escamez et al., 2005, Eur Arch Otorhinolaryngol; 262:507-511). While one third of BPPV cases in young people can be attributed to head trauma/injury, idiopathic BPPV cases are much more common in middle-aged and older people, which also tend to be recurrent (Cohen et al., 2004, J Otorhinolaryngol Relat Spec; 66:11-15; and Ogun et al., 2014, PLoS One; 9:e105546). The prevalence drastically increases at middle age and older (Ogun et al., 2014, Menopause; 21:886-889; and Von Brevern et al., 2007, J Neurol Neurosurg Psychiatry; 78(7):710-5).

Currently, no medication is available to treat or prevent BPPV. And, although BPPV is treatable with maneuvers to reposition the dislocated otoconia in the utricle, there is a high recurrence rate of about 30% within the first year and 50% after 5 years (Hain et al., 2000, Arch Otolaryngol Head Neck Surg; 126(5):617-22; Nunez et al., 2000, Otolaryngol Head Neck Surg; 122(5):647-52; and Sakaida et al., 2003, Neurology; 60(9):1532-4). Surgical options, including labyrinthectomy and semicircular canal occlusion, can be used for such severe recurrent cases. However, those procedures are rarely used due to their complications and destructiveness.

Therefore, there is a need for a better understanding of the molecular and cellular events contributing to benign paroxysmal positional vertigo and a need for the development of improved and alternate therapies for the treatment of BPPV, especially for cases of BPPV that are resistant to repositioning maneuvers.

### SUMMARY OF THE INVENTION

The present disclosure includes a method of diagnosing Benign Paroxysmal Positional Vertigo (BPPV) in a subject, the method including determining that at least one allele of the human protocadherin gamma A10 (PCDHGA10) gene in the subject's genome includes a variation encoding a premature stop codon within intracellular/cytoplasmic domain of the short isoform the human PCDHGA10 polypeptide. In some aspects, the variation encoding a premature stop codon truncates the intracellular/cytoplasmic domain of the short isoform the human PCDHGA10 polypeptide after about amino acid residue 812. In some aspects, the variation encoding a premature stop codon includes an insertion mutation. In some aspects, the variation encoding a premature stop codon includes NM 032090.1:c.2476_2477dup in the protocadherin gamma A10 gene (PCDHGA10). In some aspects, the variation encoding a premature stop codon includes NM_032090.1:c.2477dup in the protocadherin gamma A10 gene (PCDHGA10). In some aspects, the variation encoding a premature stop codon includes NM_032090.1:c.2467_2468insA (p.Lys823_Lys824fs) in the protocadherin gamma A10 gene (PCDHGA10). In some aspects, the variation encoding a premature stop codon includes the SNP allele Rs113784532, rs369101565, rs752029921, or rs750612188.

The present disclosure includes a method of diagnosing Benign Paroxysmal Positional Vertigo (BPPV) in a subject, the method including genotyping the subject or having the subject genotyped and determining that at least one allele of the human protocadherin gamma A10 (PCDHGA10) gene in the subject's genome includes a variation encoding a premature stop codon within intracellular/cytoplasmic domain of the short isoform the human PCDHGA10 polypeptide. In some aspects, the variation encoding a premature stop codon truncates the intracellular/cytoplasmic domain of the short isoform the human PCDHGA10 polypeptide after about amino acid residue 812. In some aspects, the variation encoding a premature stop codon includes an insertion mutation. In some aspects, the variation encoding a premature stop codon includes NM_032090.1:c.2476_2477dup in the protocadherin gamma A10 gene (PCDHGA10). In some aspects, the variation encoding a premature stop codon includes NM_032090.1:c.2477dup in the protocadherin gamma A10 gene (PCDHGA10). In some aspects, the variation encoding a premature stop codon includes NM_032090.1:c.2467_2468insA (p.Lys823_Lys824fs) in the protocadherin gamma A10 gene (PCDHGA10). In some aspects, the variation encoding a premature stop codon includes the SNP allele Rs113784532, rs369101565, rs752029921, or rs750612188.

The present disclosure includes a method diagnosing Benign Paroxysmal Positional Vertigo (BPPV) in a subject, the method including determining that at least one allele of the human Caspase (CASP10) gene in the subject's genome includes a single nucleotide variation at a position encoding amino acid residue 410. In some aspects, the variation includes a valine to isoleucine substitution at position 410. In some aspects, the variation includes NM_032974.5:c1228G>A. In some aspects, the variation includes the SNP allele rs13010627.

The present disclosure includes a method of diagnosing Benign Paroxysmal Positional Vertigo (BPPV) in a subject, the method including genotyping the subject or having the subject genotyped and determining that at least one allele of the human Caspase 10 (CASP10) gene in the subject's genome includes a single nucleotide variation at a position encoding amino acid residue 410. In some aspects, the variation includes a valine to isoleucine substitution at position 410. In some aspects, the variation includes NM_032974.5:c1228G>A. In some aspects, the variation includes the SNP allele rs13010627.

Regarding any of the methods described herein, in some embodiments the variation may be identified by genomic sequencing. In other embodiments, the variation may be identified from previously obtained genomic sequencing data

With any of the methods described herein, the variation may be identified by polymerase chain reaction (PCR). In some aspects, the method further includes sequencing the PCR product. In some aspects, a variation encoding a premature stop codon within intracellular/cytoplasmic domain of the short isoform the human PCDHGA10 polypeptide may be identified with a forward primer includes AAGAGTCACCTGATCTTCCC (SEQ ID NO:5), and a reverse primer includes ACACTGGAGTAAAAACCAATCTTTT (SEQ ID NO:6). In some aspects, encoding a premature stop codon within the intracellular/cytoplasmic domain of the short isoform the human PCDHGA10 polypeptide may be identified with a forward primer includes ACACTCGAGCTGTGAGAAAAAAGATCC-3′ (SEQ ID NO:7) and a reverse primer includes ACATCTAGATTTGGGCTCAAGCACAACG (SEQ ID NO:8). In some aspects, a variation includes a single nucleotide variation at position 410 of the human Caspase 10 (CASP10) gene may be identified with a forward primer includes AGGCCCTCATTCCCATTCG (SEQ ID NO:9) and a reverse primer includes TATACCAGCTGCCTTCCTC (SEQ ID NO:10).

With any of the methods described herein, the method may further include treating the subject with maneuvers to reposition the dislocated otoconia in the utricle, labyrinthectomy, and/or and semicircular canal occlusion. In some aspects, the subject has been resistant to treatment with maneuvers to reposition the dislocated otoconia in the utricle.

With any of the methods described herein, the method may further include treating the subject with an agent that inhibits the aggregation of neural proteins. In some aspects, the subject has been resistant to treatment with maneuvers to reposition the dislocated otoconia in the utricle. In some aspects, the inhibitor of the aggregation of neural proteins inhibits the aggregation of proteins in the vestibular ganglia.

The present disclosure includes a method of treating a subject with Benign Paroxysmal Positional Vertigo (BPPV), the method including administering to the subject an inhibitor of the aggregation of neural proteins. In some aspects, the subject has been resistant to treatment with maneuvers to reposition the dislocated otoconia in the utricle. In some aspects, the inhibitor of the aggregation of neural proteins inhibits the aggregation of proteins in the vestibular ganglia.

The present disclosure includes a method of preventing Benign Paroxysmal Positional Vertigo (BPPV) in a subject, the method including administering to the subject an inhibitor of the aggregation of neural proteins. In some aspects, the inhibitor of the aggregation of neural proteins inhibits the aggregation of proteins in the vestibular ganglia.

The present disclosure includes a CRISPR/cas9 gene edited mouse including at least one allele of the human protocadherin gamma A10 (PCDHGA10) gene edited to includes a variation encoding a premature stop codon within intracellular/cytoplasmic domain of the short isoform the human PCDHGA10 polypeptide. In some aspects, the variation encoding a premature stop codon truncates the intracellular/cytoplasmic domain of the short isoform the human PCDHGA10 polypeptide after about amino acid residues 812. In some aspects, the variation encoding a premature stop codon includes an insertion mutation. In some aspects, the variation encoding a premature stop codon includes: NM_032090.1:c.2476_2477dup in the protocadherin gamma A10 gene (PCDHGA10); NM_032090.1:c.2477dup in the protocadherin gamma A10 gene (PCDHGA10); NM_032090.1:c.2467_2468insA (p.Lys823_Lys824fs) in the protocadherin gamma A10 gene (PCDHGA10); or the SNP allele Rs113784532, rs369101565, rs752029921, or rs750612188.

The present disclosure includes a method of identifying an agent that inhibits the aggregation of proteins in neural cells, the method including: administering an agent to a CRISPR/cas9 gene edited mouse including at least one allele of the human protocadherin gamma A10 (PCDHGA10) gene edited to includes a variation encoding a premature stop codon within intracellular/cytoplasmic domain of the short isoform the human PCDHGA10 polypeptide assaying protein aggregation in neural cells in the mouse administered the agent; assaying protein aggregation in neural cells in the mouse to which an agent has not been administered; and comparing protein aggregation in neural cells in the mouse administered the agent to protein aggregation in neural cells in the mouse to which an agent has not been administered. In some aspects, the variation encoding a premature stop codon truncates the intracellular/cytoplasmic domain of the short isoform the human PCDHGA10 polypeptide after about amino acid residues 812. In some aspects, the variation encoding a premature stop codon includes an insertion mutation. In some aspects, the variation encoding a premature stop codon includes: NM_032090.1:c.2476_2477dup in the protocadherin gamma A10 gene (PCDHGA10); NM_032090.1:c.2477dup in the protocadherin gamma A10 gene (PCDHGA10); NM_032090.1:c.2467_2468insA (p.Lys823_Lys824fs) in the protocadherin gamma A10 gene (PCDHGA10); or the SNP allele Rs113784532, rs369101565, rs752029921, or rs750612188.

The present disclosure includes an antibody that specifically binds to a short isoform of the human protocadherin gamma A10 (PCDHGA10) polypeptide encoded by a variation encoding a premature stop codon within intracellular/cytoplasmic domain of the human PCDHGA10 polypeptide, wherein the antibody does not specifically bind to the wild type short isoform of the human protocadherin gamma A10 (PCDHGA10) polypeptide and does not specifically bind to the wild type long isoform of the human protocadherin gamma A10 (PCDHGA10) polypeptide. In some aspects, the variation encoding a premature stop codon truncates the intracellular/cytoplasmic domain of the short isoform the human PCDHGA10 polypeptide after about amino acid residues 812. In some aspects, the variation a premature stop codon includes an insertion mutation. In some aspects, the variation encoding a premature stop codon includes: NM_032090.1:c.2476_2477dup in the protocadherin gamma A10 gene (PCDHGA10); NM_032090.1:c.2477dup in the protocadherin gamma A10 gene (PCDHGA10); NM_032090.1:c.2467_2468insA (p.Lys823_Lys824fs) in the protocadherin gamma A10 gene (PCDHGA10); or the SNP allele Rs113784532, rs369101565, rs752029921, or rs750612188.

The present disclosure includes a method of monitoring protein aggregation in neural cells, the method including contacting the neural cells with an antibody that specifically binds to a short isoform of the human protocadherin gamma A10 (PCDHGA10) polypeptide encoded by a variation encoding a premature stop codon within intracellular/cytoplasmic domain of the human PCDHGA10 polypeptide, wherein the antibody does not specifically bind to the wild type short isoform of the human protocadherin gamma A10 (PCDHGA10) polypeptide and does not specifically bind to the wild type long isoform of the human protocadherin gamma A10 (PCDHGA10) polypeptide. In some aspects, the variation encoding a premature stop codon truncates the intracellular/cytoplasmic domain of the short isoform the human PCDHGA10 polypeptide after about amino acid residues 812. In some aspects, the variation a premature stop codon includes an insertion mutation. In some aspects, the variation encoding a premature stop codon includes: NM_032090.1:c.2476_2477dup in the protocadherin gamma A10 gene (PCDHGA10); NM_032090.1:c.2477dup in the protocadherin gamma A10 gene (PCDHGA10); NM_032090.1:c.2467_2468insA (p.Lys823_Lys824fs) in the protocadherin gamma A10 gene (PCDHGA10); or the SNP allele Rs113784532, rs369101565, rs752029921, or rs750612188.

The present disclosure includes a method of identifying an agent that inhibits the aggregation of proteins in the vestibular ganglia, the method including: culturing in vitro neural cells expressing a human protocadherin gamma A10 (PCDHGA10) polypeptide encoded by a PCDHGA10 gene including premature stop codon within intracellular/cytoplasmic domain of the short isoform the human PCDHGA10 polypeptide; contacting the cells with the agent; and assaying for protein aggregation in the cells. In some aspects, the premature stop codon truncates the intracellular/cytoplasmic domain of the short isoform the human PCDHGA10 polypeptide after about amino acid residues 812. In some aspects, the premature stop codon includes an insertion mutation. In some aspects, the premature stop codon includes: NM_032090.1:c.2476_2477dup in the protocadherin gamma A10 gene (PCDHGA10); NM_032090.1:c.2477dup in the protocadherin gamma A10 gene (PCDHGA10); NM_032090.1:c.2467_2468insA (p.Lys823_Lys824fs) in the protocadherin gamma A10 gene (PCDHGA10); or the SNP allele Rs113784532, rs369101565, rs752029921, or rs750612188. In some aspects, protein aggregation in the cells is assayed with an antibody that specifically binds to a short isoform of the human protocadherin gamma A10 (PCDHGA10) polypeptide encoded by a variation encoding a premature stop codon within intracellular/cytoplasmic domain of the human PCDHGA10 polypeptide, wherein the antibody does not specifically bind to the wild type short isoform of the human protocadherin gamma A10 (PCDHGA10) polypeptide and does not specifically bind to the wild type long isoform of the human protocadherin gamma A10 (PCDHGA10) polypeptide. In some aspects, the variation encoding a premature stop codon truncates the intracellular/cytoplasmic domain of the short isoform the human PCDHGA10 polypeptide after about amino acid residues 812. In some aspects, the variation encoding a premature stop codon includes an insertion mutation. In some aspects, the variation encoding a premature stop codon includes: NM_032090.1:c.2476_2477dup in the protocadherin gamma A10 gene (PCDHGA10); NM_032090.1:c.2477dup in the protocadherin gamma A10 gene (PCDHGA10); NM_032090.1:c.2467_2468insA (p.Lys823_Lys824fs) in the protocadherin gamma A10 gene (PCDHGA10); or the SNP allele Rs113784532, rs369101565, rs752029921, or rs750612188.

The above summary of the present disclosure is not intended to describe each disclosed embodiment or every implementation of the present disclosure. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF THE FIGURES

FIG. 1. Pedigrees of the 12 BPPV families selected for WES (+UTR). Circle and square symbols represent female and male individuals, respectively. Symbols with slashes indicate deceased individuals. Filled triangles indicate the probands of each family. BPPV affected and unaffected family members are denoted in filled and unfilled symbols, respectively. Pentagrams indicate the family members selected for whole genome sequencing (WES). FB: familial BPPV.

FIG. 2. Tiered selection of variants from WES of 12 BPPV families.

FIGS. 3A-3D. Frameshift mutation causes premature stop, truncating the PCDHGA10 short isoform. FIG. 3A: Verification of the NM_032090.1:c.2476_2477dup in PCDHGA10 by Sanger sequencing. FIG. 3B: Protein structures of PCDHGA10 long, short, and mutant isoforms. EC, extracellular; TM, transmembrane; Const: constant domain. FIG. 3C: Fluorescent immunostaining shows that wildtype PCDHGA10 has no large aggregates in the cytosol of non-BPPV samples at young ages (the vestibular ganglia is shown). FIG. 3D: PCDHGA10 with the rs113784532 mutation forms large intracellular aggregates in BPPV samples even at young ages. Arrows indicate aggregates. M30 and M32, male at 30 and 32 years of age, respectively. Scale bar, 25 μm.

FIG. 4. Verification of the c.1228G>A mutation in CASP10 (NM_032974.5) by Sanger sequencing. Sequencing of the CASP10 gene showed some BPPV affected cases to be heterozygous for the loci and their unaffected family members to be homozygous for the wild-type CASP10.

FIGS. 5A-5D. Fluorescent immunostaining of Pcdhga10 in the murine vestibule at 2 months of age. Arrows indicate hair cells, arrowheads transitional epithelial cells and hollow arrow vestibular ganglia. Scale bar, 25μ.m. FIG. 5A: immunostaining of the utricle. FIG. 5B: immunostaining of the crista. FIG. 5C: immunostaining of the saccule. FIG. 5D: immunostaining of the ganglia.

FIGS. 6A-6G. Localization of the long isoform of Pcdhga10 mRNA in mouse vestibular ganglia at different ages using fluorescent in situ hybridization. At P0 (FIG. 6A) and 2M (FIG. 6C), the fluorescent signals of the antisense probe of the long isoform are absent (or similar to that in the negative controls at the same ages) (FIG. 6B and FIG. 6D), whereas at 18M (FIG. 6E), the fluorescent signal of the antisense probe is much stronger than the negative control (FIG. 6F). FIG. 6G: graphical representation of fluorescence intensity at P0, 2M, and 18M. P0, postnatal day 0; 2M and 18M, 2 and 18 months old, respectively. VeG, vestibular ganglia. Scale bar, 25μ.m. *** and ###denote p<0.001 when the 18M group is compared with P0 and 2M, respectively (n=4 mice/group).

FIGS. 7A-7G. Localization of the short isoform of Pcdhga10 mRNA in mouse vestibular ganglia at different ages using fluorescent in situ hybridization. Fluorescent signals of the antisense probe of the short isoform increase with age, whereas signals of the negative controls are at background levels. FIG. 7G: graphical representation of fluorescence intensity at P0, 2M, and 18M. P0, postnatal day 0(FIG. 7A and FIG. 7B); 2M (FIGS. 7C and 7D) and 18M (FIG. 7E and FIG. 7F), 2 and 18 months old, respectively. VeG, vestibular ganglia. Scale bar, 25μ.m. *** denotes p<0.001 when compared with P0, #denotes p<0.05 when the 2M group is compared with 18M (n=4 mice/group).

FIGS. 8A-8D. Fluorescent immunostaining of PCDHGA10 and co-localization with the autophagy protein p62 in human vestibular ganglia. With FIGS. 68A and 8C, no large PCDHGA10 aggregates are seen in the cytosol of non-BPPV samples. With FIGS. 8B and 8D, mutant PCDHGA10 forms large intracellular aggregates in the vestibular ganglia of BPPV samples at young (FIG. 8B) and old ages (FIG. 8D). In FIG. 8B, p62 has not accumulated yet at young ages. In FIG. 8D, p62 co-accumulates with PCDHGA10 in large aggregates at old ages. Arrows indicate aggregates. M30 and 32, males at 30 and 32 years old, respectively. M70 and F70, male and female at 70 years old, respectively. Scale bar, 25μ.m.

FIGS. 9A and 9B. Scanning electron microscopy of otoconia in Pcdhga10 null mice (FIG. 9B) and litter-mate wildtype controls (FIG. 9A). Postnatal 15 day-old mice are shown. The mice are in the CBA/CaJ genetic background.

FIG. 10. Proteostasis pathways: cellular machinery for garbage disposal. See Cook et al., 2012, Cold Spring Harb Perspect Med; 2:a009423; and Tanaka and Matsuda, 2014, Biochim Biophys Acta; 1843:197-204.

FIGS. 11A-11C. FIG. 11A shows the Homo sapiens protocadherin gamma A10 (PCDHGA10) amino acid sequence. NCBI Reference Sequence: NM_032090.1 (SEQ ID NO: 1). FIG. 11B and FIG. 11C show the Homo sapiens protocadherin gamma A10 (PCDHGA10) cDNA nucleotide sequence. FIG. 11C is a continuation of FIG. 11B. NCBI Reference Sequence: NM_032090.1 (SEQ ID NO: 2).

FIGS. 12A-12C. FIG. 12A shows the Homo sapiens caspase 10 (CASP10) amino acid sequence. NCBI Reference Sequence: NM_032974.5 (SEQ ID NO: 3). FIG. 12B and FIG. 12C show the Homo sapiens caspase 10 (CASP10) cDNA nucleotide sequence. FIG. 12C is a continuation of FIG. 12B. NCBI Reference Sequence: NM_032974.5 (SEQ ID NO: 4).

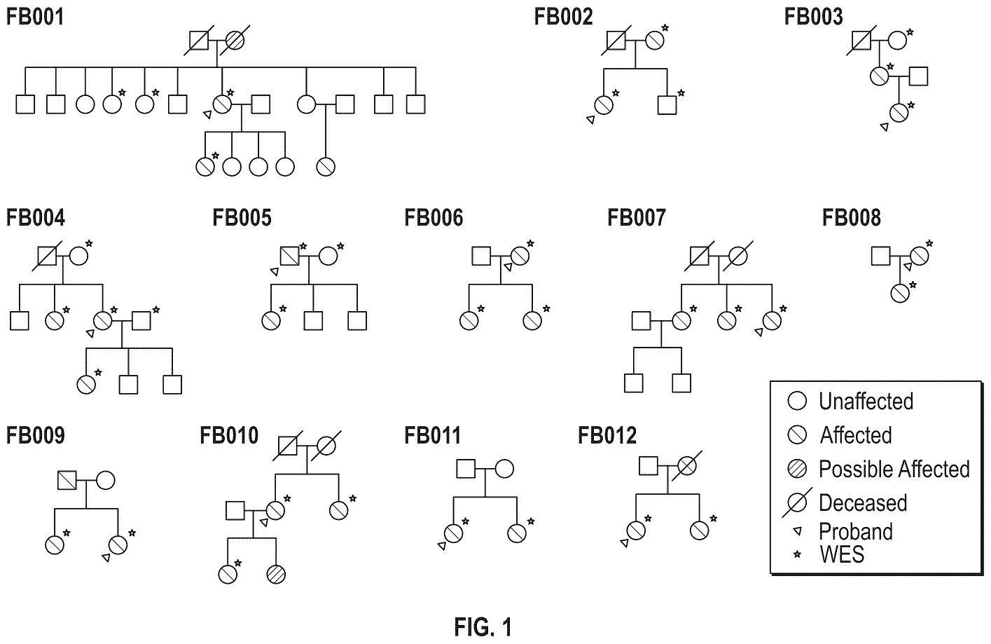

### DETAILED DESCRIPTION

### Definitions

The term “and/or” means one or all of the listed elements or a combination of any two or more of the listed elements.

The words “preferred” and “preferably” refer to embodiments of the invention that may afford certain benefits, under certain circumstances. However, other embodiments may also be preferred, under the same or other circumstances. Furthermore, the recitation of one or more preferred embodiments does not imply that other embodiments are not useful and is not intended to exclude other embodiments from the scope of the invention.

The terms “comprises” and variations thereof do not have a limiting meaning where these terms appear in the description and claims.

Unless otherwise specified, “a,” “an,” “the,” and “at least one” are used interchangeably and mean one or more than one.

Numeric ranges recited within the specification are inclusive of the numbers defining the range and include each integer within the defined range. Throughout this disclosure, various aspects of this invention are presented in a range format. It should be understood that the description in range format is merely for convenience and brevity and should not be construed as an inflexible limitation on the scope of the invention. Accordingly, the description of a range should be considered to have specifically disclosed all the possible sub-ranges, fractions, and individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6, and decimals and fractions, for example, 1.2, 3.8, 1½, and 4¾ This applies regardless of the breadth of the range. Notwithstanding that the numerical ranges and parameters setting forth the broad scope of the invention are approximations, the numerical values set forth in the specific examples are reported as precisely as possible. All numerical values, however, inherently contain a range necessarily resulting from the standard deviation found in their respective testing measurements.

For any method disclosed herein that includes discrete steps, the steps may be conducted in any feasible order. And, as appropriate, any combination of two or more steps may be conducted simultaneously.

Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present disclosure. At the very least, and not as an attempt to limit the doctrine of equivalents to the scope of the claims, each numerical parameter should at least be construed in light of the number of reported significant digits and by applying ordinary rounding techniques. The term “about,” as used herein, refers to variation in the numerical quantity that can occur, for example, through typical measuring techniques and equipment, with respect to any quantifiable variable, including, but not limited to, molecular weight, mass, sequence identity, percent homology, pH, temperature, and time. Further, given solid and liquid handling procedures used in the real world, there is certain inadvertent error and variation that is likely through differences in the manufacture, source, or purity of the ingredients used to make the compositions or carry out the methods and the like.

All headings are for the convenience of the reader and should not be used to limit the meaning of the text that follows the heading, unless so specified.

### Benign Paroxysmal Positional Vertigo

Benign Paroxysmal Positional Vertigo (BPPV) is the most common cause of vertigo in humans. It is a disorder with heterogeneous nongenetic (Cohen et al., 2004, J Otorhinolaryngol Relat Spec; 66:11-15; Ogun et al., 2014, Menopause; 21:886-889; Hughes and Proctor, 1997, Laryngoscope; 107:607-613; Modugno et al., 2000, Med Hypotheses; 54:614-615; and Papi et al., 2010, Thyroid; 20:237-238) and genetic causes (Ogun et al., 2014, PLoS One; 9:e105546; Gizzi et al., 2014, Int J Neurosci; 125(8):593-6; and Lee et al., 2006, Hum Mol Genet; 15:251-258). There is a familial predisposition in BPPV occurrence (Ogun et al., 2014, PLoS One; 9:e105546). Indeed, an earlier genetic analysis of a three-generation family in which multiple family members developed BPPV mapped the trait to chromosome 15 (Katsarkas, 1999, Acta Otolaryngol; 119:745-749; and Gizzi et al., 2014, Int J Neurosci; 125(8):593-6). Another genetic study (Lee et al., 2006, Hum Mol Genet; 15:251-258) mapped recurrent vertigo to chromosome 22q12. While most BPPV families suggest dominant inheritance with reduced penetrance, using autosomal dominant and recessive models with reduced penetrance, the above papers showed linkage in the same regions of chromosomes 15 and 22, respectively. However, the underlying genes were not identified and the molecular etiology of BPPV remains unknown.

Genetic Variations Associated with Idiopathic BPPV

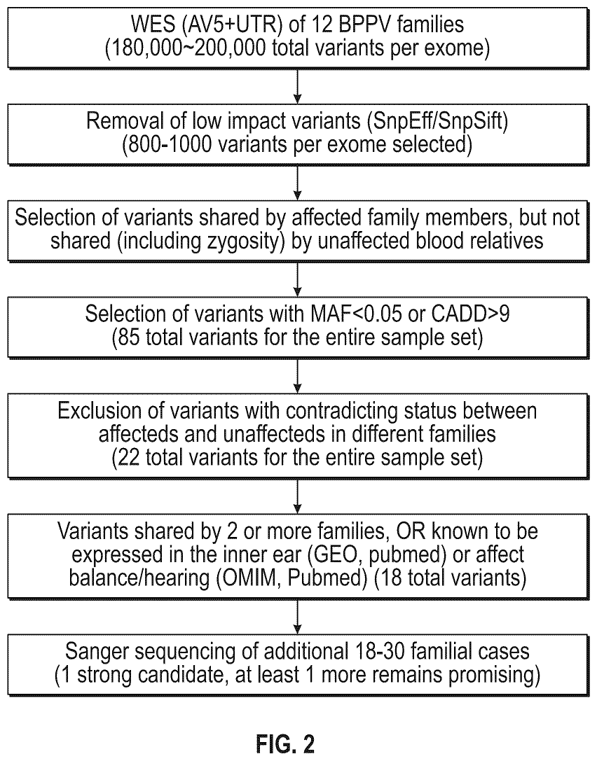

With the present disclosure multiple genetic variations associated with idiopathic BPPV in humans have been identified. These genetic variants include a genetic variant of the human protocadherin gamma A10 (PCDHGA10) gene mapping to chromosome 5 and a genetic variant of the human Caspase 10 (CASP10) gene, mapping to chromosome 2. As described in Example 1 and shown in FIG. 2, whole exome sequencing of familial cases of BPPV was performed, using 12 families of non-Hispanic white from the US Midwest, each with 2-3 affecteds and 0-2 of unaffected blood relatives for a total of 28 affecteds and 14 unaffecteds. Each exome had at least 97.6% covered at >10×, and 93.0% at >20×. After removal of synonymous and low impact intronic variants, those meeting the following criteria were selected for Sanger sequencing of additional samples: (1) variants are shared by affected but not by unaffected family members, and variants versus phenotypes are consistent between affecteds and unaffecteds within a family and among different families; (2) variants have a minor allele frequency of lower than 0.05, or a CADD (combined annotation dependent depletion, a tool for scoring the deleteriousness of variants in the human genome) score above 9; (3) variants are shared by 2 or more families, known to be expressed in the inner ear (GEO, Pubmed) or known to affect balance/hearing (OMIM, Pubmed). Using this tiered selection process, a total of 18 variants were selected for Sanger sequencing of additional 18-29 BPPV families.

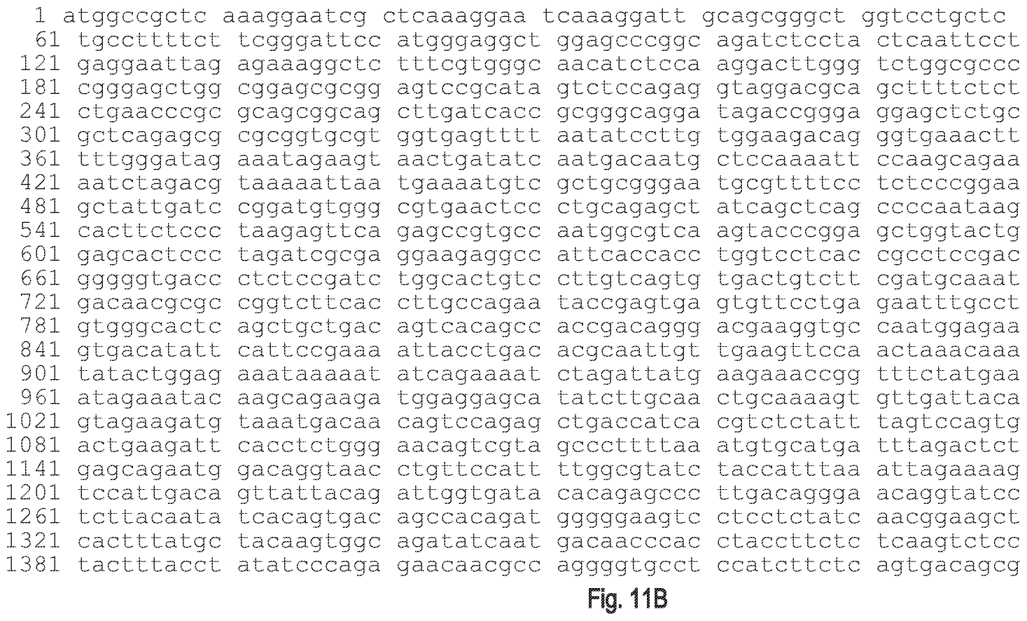

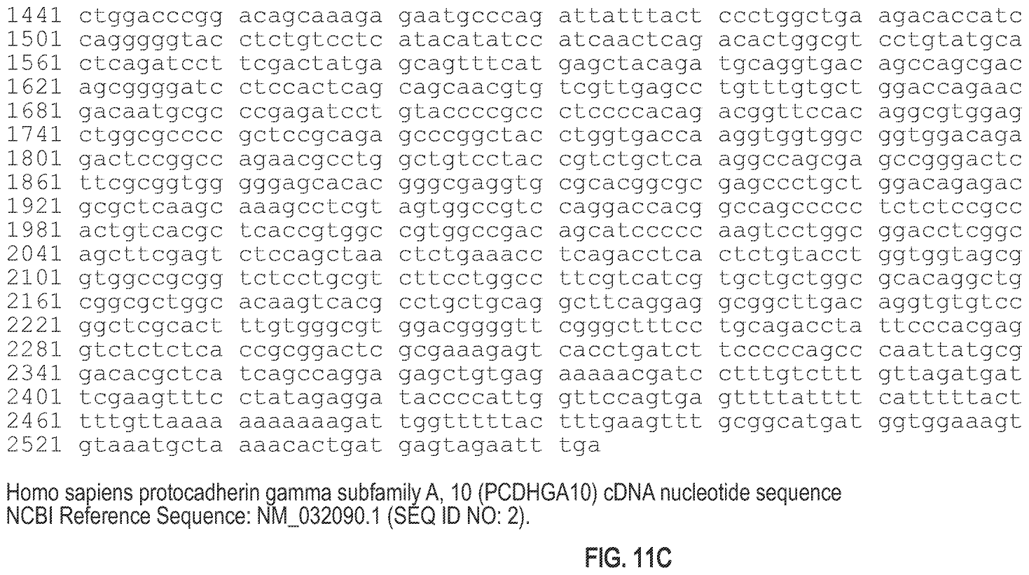

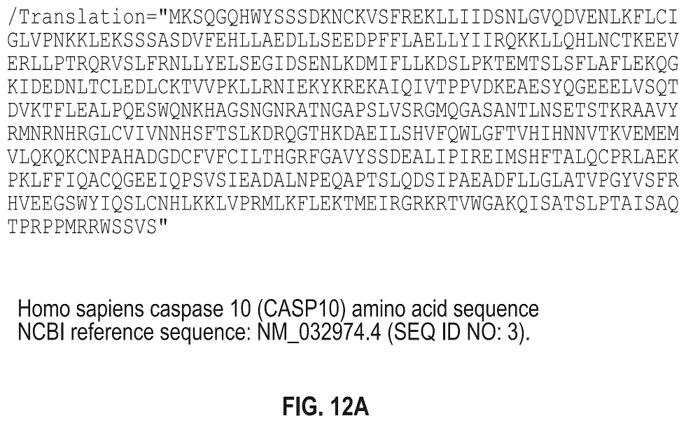

A first genetic variant that is strongly associated with familial cases of recurrent BPPV is a rare variant in the short isoform of the human protocadherin gamma A10 (PCDHGA10) gene. As described in more detail in Example 1, this genetic variant (also referred to herein as a “mutation”) accounts for 45% of the familial cases of BPPV studied. The short isoform of the human PCDHGA10 gene is also referred to as Homo sapiens protocadherin gamma subfamily A, 10 (PCDHGA10), transcript variant 2, mRNA and is identified by NCBI Reference Sequence: NM_032090.1. FIG. 11A shows the NCBI Reference Sequence: NM_032090.1 Homo sapiens protocadherin gamma A10 (PCDHGA10) amino acid sequence (SEQ ID NO: 1). FIG. 11B shows the NCBI Reference Sequence: NM_032090.1 Homo sapiens protocadherin gamma A10 (PCDHGA10) cDNA nucleotide sequence (SEQ ID NO: 2).

PCDHGA10 belongs to the γ-subfamily of the protocadherins (PCDH) super family of 70 genes. PCDHs are concentrated in neurons and synapses. Fifty-eight of the PCDHs are tandemly arrayed in three clusters (α, β, and γ) on human chromosome 5 and mouse chromosome 18. Each cluster contains multiple “variable exons” encoding the extracellular, transmembrane, and cytoplasmic domains of individual isoforms. Within the PCDH-α and γ clusters, each “variable exon” is transcribed from its own promoter and spliced to constant exons that encode a shared C-terminal constant domain (Obata et al., 1998, Cell Adhes Commun; 6:323-333; Sugino et al., 2000, Genomics; 63:75-87; Tasic et al., 2002, Mol Cell; 10:21-33; and Wang et al., 2002, Genes Dev; 16:1890-1905), much like the clusters encoding immunoglobulins (Ig) and Tcell receptors. The constant domain of PCDHs is identical within the same cluster, and highly conserved between clusters.

PCDHGA10 is a member of the protocadherin gamma gene cluster, one of three related clusters tandemly linked on chromosome five. These gene clusters have an immunoglobulin-like organization, suggesting that a novel mechanism may be involved in their regulation and expression. The gamma gene cluster includes genes divided into subfamilies. Subfamily A contains 12 genes, subfamily B contains 7 genes and 2 pseudogenes, and the more distantly related subfamily C contains 3 genes. The tandem array of 22 large, variable region exons are followed by a constant region, containing 3 exons shared by all genes in the cluster. Each variable region exon encodes the extracellular region, which includes 6 cadherin ectodomains and a transmembrane region. The constant region exons encode the common cytoplasmic region. These neural cadherin-like cell adhesion proteins most likely play a critical role in the establishment and function of specific cell-cell connections in the brain.

Depending on the family member and interacting molecules, γ-PCDHs are important for synaptic formation and maturation, spatial patterning of axons and dendrites, dendritic pruning and self-avoidance, and neuronal survival (Garrett and Weiner, 2009, J Neurosci; 29:11723-11731; Kostadinov and Sanes, 2015, Elife; 4 (DOI: 10.7554/eLife.08964); and Molumby et al., 2017, Cell Rep; 18:2702-2714). Unlike classical cadherins, which are present at the cell surface, γ-PCDHs have a prominent intracellular presence in neurons, particularly in endolysosomes (Wang et al., 2002, Neuron; 36:843-854; Phillips et al., 2003, J Neurosci; 23:5096-5104; and Fernandez-Monreal et al., 2010, Eur J Neurosci; 32:921-931). The variable domains of γ-PCDHs are the most important contributor to localizing the protein to endolysosomes (O'Leary et al., 2011, Mol Blot Cell; 22:4362-4372; and Shonubi et al., 2015, BMC Cell Biol; 16:28). There are not many studies on PCDHs, and most are focused on their roles in development. Findings in mutant mice suggest that the predominant role of γ-PCDHs is neuronal survival (Wang et al., 2002, Neuron; 36:843-854). However, little is known about their roles in adulthood, and details regarding their function in neurons are lacking. Nothing is known about that of PCDHGA10.

With the present disclosure, the strongest genetic variant associated with familial cases of recurrent BPPV is a rare variant in the short isoform of the PCDHGA10 gene. This genetic variant encodes a premature stop codon within intracellular/cytoplasmic domain of the short isoform the human PCDHGA10 polypeptide. The premature stop codon may be located within the intracellular/cytoplasmic domain of the short isoform the human PCDHGA10 polypeptide. The premature stop codon may truncate the intracellular/cytoplasmic domain of the short isoform the human PCDHGA10 polypeptide after about amino acid residue 812. The premature stop codon may be the result of an insertion mutation.

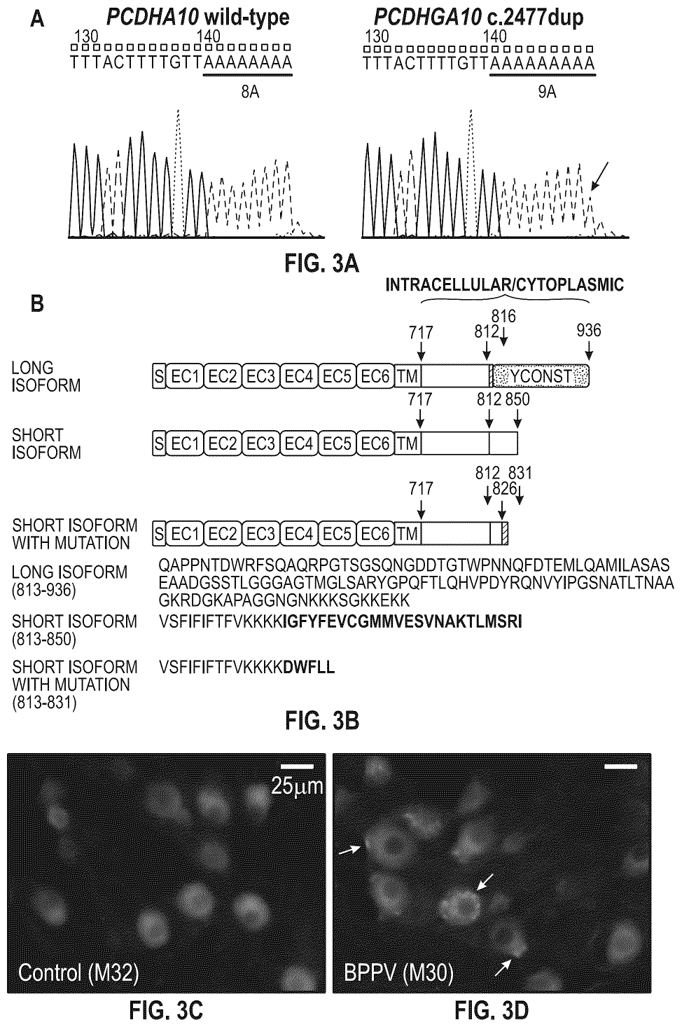

In some embodiments, the genetic variation encoding the premature stop codon is NM_032090.1:c.2476_2477dup in the protocadherin gamma A10 gene (PCDHGA10). In some embodiments, the genetic variation encoding a premature stop codon is NM_032090.1:c.2477dup in the protocadherin gamma A10 gene (PCDHGA10). In some embodiments, the genetic variation encoding the premature stop codon is NM_032090.1:c.2467_2468insA (p.Lys823_Lys824fs) in the protocadherin gamma A10 gene (PCDHGA10). See Table 2. This frameshift mutation causes a premature stop, truncating the PCDHGA10 short isoform (FIG. 3).

In some embodiments, the genetic variation encoding the premature stop codon includes the single-nucleotide polymorphism (SNP) allele Rs113784532, rs369101565, rs752029921, or rs750612188. A SNP, also referred to as a single nucleotide variant (SNV), is a location in the genome that is known to vary between individuals. The Single Nucleotide Polymorphism Database is a free public archive for genetic variation within and across different species developed and hosted by the National Center for Biotechnology Information (NCBI) in collaboration with the National Human Genome Research Institute (NHGRI). And an rs number is an accession number used by researchers and databases to refer to specific SNPs. It stands for Reference SNP cluster ID.

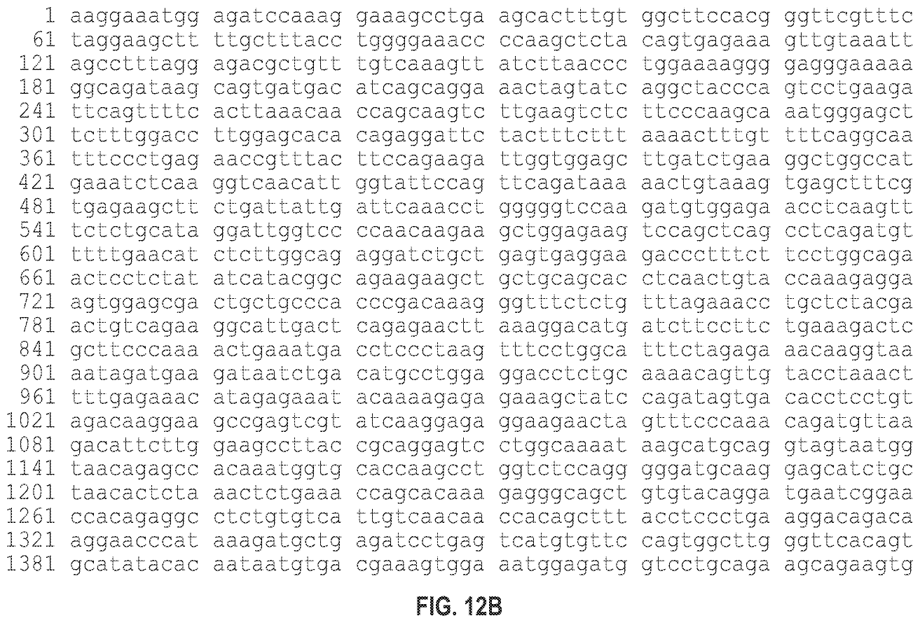

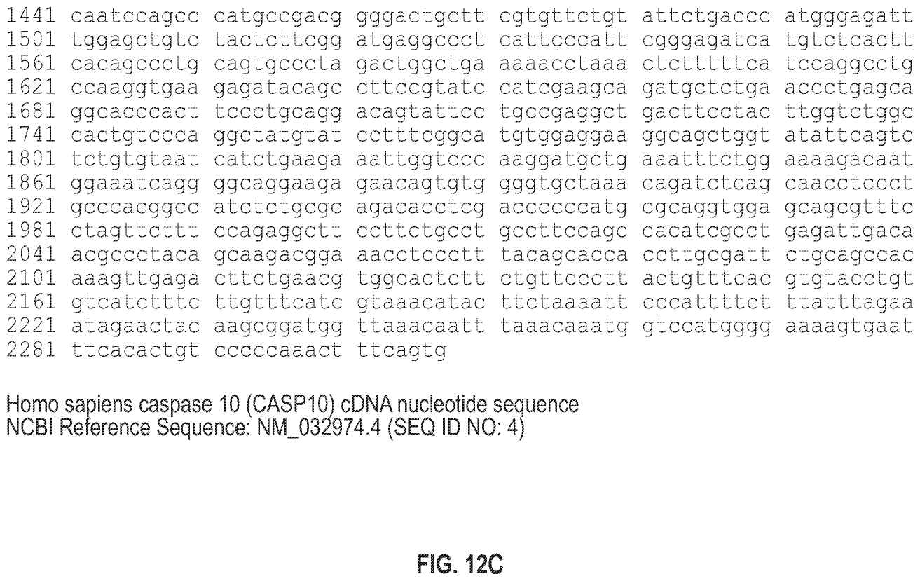

Using the methodology described in Example 1, and as described in more detail in Example 2, a second genetic variant associated with familial cases of recurrent BPPV lies in the Caspase-10 gene (CASP10, also known as MHC4 or FLICE2) located in human chromosome 2q33. The Homo sapiens caspase 10 (CASP10) gene is identified by NCBI Reference Sequence NM_032974.5. On Jun. 2, 2019, the sequence version NM_032974.5 replaced NM_032974.4. FIG. 12A shows the Homo sapiens caspase 10 (CASP10) amino acid sequence (SEQ ID NO: 3). FIG. 12B shows the Homo sapiens caspase 10 (CASP10) cDNA nucleotide sequence (SEQ ID NO: 4).

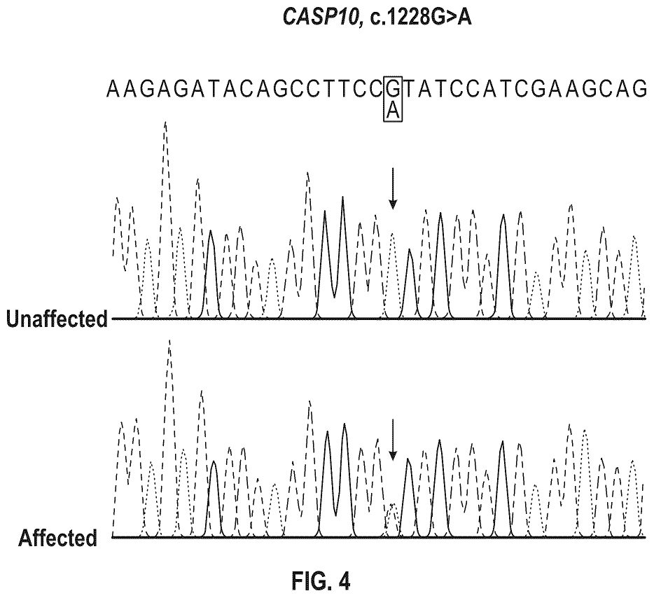

The Caspase-10 gene variant includes a single nucleotide variation at a position encoding amino acid residue 410. In some embodiments, the variation is a valine to isoleucine substitution at amino acid position 410. In some embodiments, the variation is NM_032974.5:c1228G>A. In some embodiments, the variation includes the SNP allele rs13010627. This variant includes the missense variant rs13010627 (NM_032974.5:c.1228G>A) in the apoptosis gene Caspase 10 (CASP10). This mutation results in a valine to isoleucine substitution at position 410 in CASP10 isoform 2. As described in example 2, 11 out of 57 affected carried this heterozygous variant, whereas the unaffected family members were all homozygous for the wild-type allele (G/G) (FIG. 4).

The protein product CASP10 is a cysteine protease, and the variant is located in the p17 large subunit of the protease. Like its paralogue caspase-8, CASP10 is recognized as an initiator caspase in death receptor apoptotic pathway (Wang et al., 2001, Proc Natl Acad Sci USA; 98(24):13884-8). This gene encodes a protein which is a member of the cysteine-aspartic acid protease (caspase) family. Sequential activation of caspases plays a central role in the execution-phase of cell apoptosis. Caspases exist as inactive proenzymes which undergo proteolytic processing at conserved aspartic residues to produce two subunits, large and small, that dimerize to form the active enzyme. This protein cleaves and activates caspases 3 and 7, and the protein itself is processed by caspase 8. Mutations in this gene are associated with type IIA autoimmune lymphoproliferative syndrome, non-Hodgkin lymphoma and gastric cancer.

The present disclosure includes methods of diagnosing Benign Paroxysmal Positional Vertigo (BPPV) in a subject by determining that at least one allele of the protocadherin gamma A10 (PCDHGA10) gene in the subject's genome is a PCDHGA10 genetic variant as described above and/or that at least one allele of the Caspase-10 (CASP10) gene in the subject's genome is a CASP10 genetic variant as described above.

Determining that a genetic variant of the protocadherin gamma A10 (PCDHGA10) gene and/or the Caspase-10 (CASP10) gene is present in a subject's genome may be accomplished by any of many available technologies. Such a determination may also be referred to as genotyping. A genetic variant may be heterozygous within the genome or homozygous within the genome.

Genetic variants may be identified by sequencing all or a portion of the genome, including sequencing by the massive parallel sequencing methods, also known as Next Generation Sequencing (NGS) or Sanger sequencing.

Genetic variants may be identified by polymerase chain reaction (PCR). In some embodiments, a resultant PCR product may be sequenced and/or restriction mapped. Such methods include, but are not limited to, Taqman assays (Applied Biosystems, Foster City, CA), iPLEX Sequenom MassArray system (Sequenom Inc., San Diego, CA), and PCR-based RFLP.

In some embodiments, the forward primer AAGAGTCACCTGATCTTCCC (SEQ ID NO:5) and the reverse primer ACACTGGAGTAAAAACCAATCTTTT (SEQ ID NO:6) may be used to identify genetic variants of within the intracellular/cytoplasmic domain of the short isoform the human PCDHGA10 polypeptide.

In some embodiments, the forward primer ACACTCGAGCTGTGAGAAAAAAG ATCC-3′ (SEQ ID NO: 7) and the reverse primer ACATCTAGATTTGGGCTCAAGCAC AACG (SEQ ID NO:8) may be used to identify genetic variants of within the intracellular/cytoplasmic domain of the short isoform the human PCDHGA10 polypeptide.

In some embodiments, the forward primer comprises AGGCCCTCATTCCCATTCG (SEQ ID NO:9) and the reverse primer TATACCAGCTGCCTTCCTC (SEQ ID NO:10) may be used to identify genetic variants of within the CASP10 gene.

In some embodiments, a genetic variation is identified from previously obtained genomic sequencing data.

### Treatment

Subjects identified to be carrying one of the genetic variations described herein associated with a heightened susceptibility to BPPV may then be appropriately treated. Such treatments include, but are not limited to, maneuvers to reposition the dislocated otoconia in the utricle, labyrinthectomy, semicircular canal occlusion, and/or the administration of an inhibitor of the protein aggregation. In some embodiments, the subject may have been resistant to treatment with maneuvers to reposition the dislocated otoconia in the utricle.

The present disclosure also includes methods of treating or preventing BPPV in individuals by administering an inhibitor of protein aggregation. Such an individual may or may not have been genotyped to identify one or more of the genetic variations/mutations described herein that are associated with a heightened susceptibility to BPPV. Such an individual may belong to a family demonstrating a familial association with BPPV.

An inhibitor of protein aggregation may inhibit aggregation of proteins in cells, such as, for example, neural cells. An inhibitor of protein aggregation may include an inhibitor of protein aggregation also used in the treatment and/or prevention of a progressive neurodegeneration disease such as Alzheimer's disease (AD), Parkinson's disease (PD), Huntington's disease (HD), and/or amyotrophic lateral sclerosis (ALS).

### Genetically Engineered Mice

The present disclosure also includes mice that have been genetically engineered to express the wild type long isoform, the wild type short isoform, and/or a mutant short isoform of the protocadherin gamma A10 (PCDHGA10) polypeptide. The short isoform of the protocadherin gamma A10 (PCDHGA10) polypeptide may be encoded by a genetic variation or mutation the encodes a premature stop codon within intracellular/cytoplasmic domain of the PCDHGA10 polypeptide. The premature stop codon may truncate the intracellular/cytoplasmic domain of the short isoform the PCDHGA10 polypeptide after about amino acid residues 812. The premature stop codon may include an insertion mutation.

The present disclosure also includes mice that have been genetically engineered to express a mutant short isoform of the human protocadherin gamma A10 (PCDHGA10) polypeptide. The short isoform is encoded by a genetic variation or mutation that encodes a premature stop codon within intracellular/cytoplasmic domain of the polypeptide. The premature stop codon truncates the intracellular/cytoplasmic domain of the short isoform after about amino acid residues 812.

In some aspects, the short isoform of the human protocadherin gamma A10(PCDHGA10) polypeptide may be encoded by the NM_032090.1:c.2476_2477dup in the protocadherin gamma A10 (PCDHGA10) gene; the NM_032090.1:c.2477dup in the protocadherin gamma A10 gene (PCDHGA10) gene; NM_032090.1:c.2467_2468insA (p.Lys823_Lys824fs) in the protocadherin gamma A10 (PCDHGA10) gene; or by the SNP alleles Rs113784532, rs369101565, rs752029921, or rs750612188.

In some aspects, the wild type long isoform, the wild type short isoform, and/or a mutant short isoform of the protocadherin gamma A10 (PCDHGA10) polypeptide is a human allele. In some aspects, the wild type long isoform, the wild type short isoform, and/or a mutant short isoform of the protocadherin gamma A10 (PCDHGA10) polypeptide is a murine allele.

The present disclosure also includes null mutations, mice that have been genetically engineered to delete or prevent expression of the long isoform and/or short isoform of the protocadherin gamma A10 (PCDHGA10) polypeptide.

In addition to other technologies, the CRISPR/Cas9 gene editing technology was used to generate such mice using the established CRISPR-Cas9 technology. See, for example, Harms et al., 2014, Curr Protoc Hum Genet; 83:15.7.1-27; Quadros et al., 2015, FEBS Open Bio; 5:191-7; and Miura et al., 2018, Nat Protoc; 13:195-215. For example, for a knockout mouse, two guide RNAs may be used for excising out nearly the entire exon 1. An oligonucleotide donor containing an in-frame stop codon is then used to join the cleaved ends. For the knock-in model, one guide RNA and one oligonucleotide donor containing the desired mutation may be used. The guide RNA, Cas9 protein and ssODN donors are commercially procured and injected into CBA/CaJ mouse strain pronuclei.

CRISPR/Cas9 mice carrying the identified human frameshift mutation rs113784532 may be used to quantify longitudinally protein aggregate formation in vivo and the survival rates of vestibular ganglia and identify functional deficits in homozygous and heterozygous mutant mice throughout lifespan. In some embodiments, PCDHGA10 knockout and knockin mutant mice, as described in Example 4, may be used.

Such genetically engineered mice may be used in studies to quantify longitudinally protein aggregate formation in vivo.

Such genetically engineered mice may be in studies to determine the approximate onset age of PCDHGA10 protein aggregation in mouse vestibular ganglia, and its association with the proteostasis/macroautophagy system.

Such genetically engineered mice may be used in studies to detect age-related protein aggregation in the peripheral sensory nervous system. Because people with Alzheimer's disease (AD) show sensorimotor deficits first, 5-15 years before the onset of cognitive decline (Albers et al., 2015, Alzheimers Dement; 11:70-98), establishing the earliest timeline in the proposed work is critical for identifying the optimal window of opportunity for treatment and/or mitigation of these types of diseases in the future. The main reason for various drugs to fail in AD treatment testing could be that by the time memory loss is severe, it is too late. Earlier treatment/prevention may prove to be much more effective.

Such genetically engineered mice may be used in assays to identify agents that inhibit the aggregation of proteins in neural cells. Such agents may be used in the treatment and/or prevention of diseases such as BPPV, Alzheimer's disease (AD), Parkinson's disease (PD), Huntington's disease (HD), and/or amyotrophic lateral sclerosis (ALS).

### Antibodies

Also described herein are antibodies that are specific for the wild type long isoform, the wild type short isoform, or a mutant short isoform of the human protocadherin gamma A10 (PCDHGA10) polypeptide. Also described herein are antibodies that are specific for the long or the short isoforms of the mouse protocadherin gamma A10 (PCDHGA10) polypeptide.

Included are antibodies that specifically bind to a short isoform of the human protocadherin gamma A10 (PCDHGA10) polypeptide encoded by a genetic variation or mutation encoding a premature stop codon within intracellular/cytoplasmic domain of the human PCDHGA10 polypeptide, wherein the antibody does not specifically bind to the wild type short isoform of the human protocadherin gamma A10 (PCDHGA10) polypeptide and does not specifically bind to the wild type long isoform of the human protocadherin gamma A10 (PCDHGA10) polypeptide. In some aspects, the premature stop codon truncates the intracellular/cytoplasmic domain of the short isoform the human PCDHGA10 polypeptide after about amino acid residues 812. In some aspects, the premature stop codon includes an insertion mutation. In some aspects, the premature stop codon is encoded by the NM_032090.1:c.2476_2477dup in the protocadherin gamma A10 (PCDHGA10) gene; the NM_032090.1:c.2477dup in the protocadherin gamma A10 gene (PCDHGA10) gene; NM_032090.1:c.2467_2468insA (p.Lys823_Lys824fs) in the protocadherin gamma A10 (PCDHGA10) gene; or by the SNP alleles Rs113784532, rs369101565, rs752029921, or rs750612188.

Such antibodies may be used in in vitro or in vivo assays of monitoring protein aggregation. Such antibodies may be used, for example, to perform non-quantitative or quantitative fluorescent immunostaining of cells, including, but not limited to vestibular ganglia from human postmortem tissues of BPPV and non-BPPV cases. Such antibodies may be used, for example, to perform non-quantitative or quantitative Western blotting. Such antibodies may be used, for example, to quantify sub-cellular distribution of the isoforms of Pcdhga10 at various ages in both humans and mice.

Antibodies of the present disclosure may “specifically bind to” or be “specific for” a particular polypeptide, or an epitope on a particular polypeptide. Such an antibody is one that binds to that particular polypeptide or epitope on a particular polypeptide without substantially binding to any other polypeptide or polypeptide epitope. Antibodies of the present disclosure can be assayed for immunospecific binding by the methods described herein and by any suitable method known in the art. The immunoassays that can be used include, but are not limited to, competitive and non-competitive assay systems using techniques such as BIAcore analysis, fluorescence activated cell sorter (FACS) analysis, immunofluorescence, immunocytochemistry, Western blots, radio-immunoassays, enzyme linked immunosorbent assay (ELISA), “sandwich” immunoassays, immunoprecipitation assays, precipitin reactions, gel diffusion precipitin reactions, immunodiffusion assays, agglutination assays, complement-fixation assays, immunoradiometric assays, fluorescent immunoassays, and protein A immunoassays. Such assays are routine and well known in the art.

An antibody of the present disclosure may be a polyclonal antibody or a monoclonal antibody. In preferred embodiments, an antibody of the present disclosure is a monoclonal antibody. The term “monoclonal antibody” or “monoclonal antibody composition,” as used herein, refers to a homogeneous population of antibodies. All the antibodies in the preparation recognize the same epitope on the target molecule and all of the monoclonal antibodies have the same affinity. As used herein the terms “monoclonal antibody” or “monoclonal antibodies” are used interchangeably.

The antibodies of the present disclosure can be of any type (such as, for example, IgG, IgE, IgM, IgD, IgA and IgY), class (such as, for example, IgG1, IgG2, IgG3, IgG4, IgA1 and IgA2) or subclass of immunoglobulin molecule. In some embodiments, the immunoglobulin is an IgG. An array of IgG, IgE, IgM, IgD, IgA, and IgY heavy chains can be paired with a light chain of the kappa or lambda form.

The antibodies of the invention can be from any animal origin, including birds and mammals. In some embodiments, the antibodies are human, murine, rat, donkey, sheep, rabbit, goat, guinea pig, camel, horse, llama, camel, or chicken antibodies.

Antibodies of the present disclosure may include dimeric, trimeric, and multimeric antibodies, bispecific antibodies, chimeric antibodies, human antibodies, humanized antibodies, recombinant antibodies, and engineered antibodies.

Entirely human antibodies may also be prepared and used in the present disclosure. Such human antibodies may be obtained, for example, from healthy subjects by obtaining a population of mixed peripheral blood lymphocytes from a human subject or isolated from human immunoglobulin libraries or from animals transgenic for one or more human immunoglobulins.

The present disclosure also includes various antibody fragments, also referred to as antigen binding fragments, which include only a portion of an intact antibody, generally including an antigen binding site of the intact antibody and thus retaining the ability to bind antigen. The techniques for preparing and using various antibody-based constructs and fragments are well known in the art. For example, fragments can be obtained via chemical or enzymatic treatment of an intact or complete antibody or antibody chain. Fragments can also be obtained by recombinant means. Examples of antibody fragments include, for example, Fab, Fab′, F(ab′)₂, Fd, Fd′, scFv (single chain Fv), single domain antibodies (dAB), linear antibodies, diabodies, and the like.

Antibodies of the present disclosure may be isolated. “Isolated,” when used to describe the various antibodies disclosed herein, means the antibody that has been identified and separated and/or recovered from a component of its natural environment. Contaminant components of its natural environment are materials that would typically interfere with diagnostic or therapeutic uses for the polypeptide, and may include enzymes, hormones, and other proteinaceous or non-proteinaceous solutes.

Antibodies of the present disclosure can be produced by an animal, cultured cell line, chemically synthesized, or recombinantly expressed. Monoclonal antibodies of the present disclosure can be expressed in mammalian cells, yeast, bacteria, or other cells under the control of appropriate promoters. Antibodies for different isoforms may be raised in different species (e.g. chicken, guinea pigs and rats) to facilitate double staining.

The antibodies of the present disclosure may be coupled directly or indirectly to a detectable marker by techniques well known in the art. A detectable marker is an agent detectable, for example, by spectroscopic, photochemical, biochemical, immunochemical, or chemical means. Useful detectable markers include, but are not limited to, fluorescent dyes, chemiluminescent compounds, radioisotopes, electron-dense reagents, enzymes, colored particles, biotin, or dioxigenin. A detectable marker often generates a measurable signal, such as radioactivity, fluorescent light, color, or enzyme activity. Antibodies conjugated to detectable agents may be used for diagnostic or therapeutic purposes. Examples of detectable agents include various enzymes, prosthetic groups, fluorescent materials, luminescent materials, bioluminescent materials, radioactive materials, positron emitting metals using various positron emission tomographies, and nonradioactive paramagnetic metal ions. The detectable substance can be coupled or conjugated either directly to the antibody or indirectly, through an intermediate such as, for example, a linker known in the art, using techniques known in the art. See, for example, U.S. Pat. No. 4,741,900, describing the conjugation of metal ions to antibodies for diagnostic use. Examples of suitable enzymes include horseradish peroxidase, alkaline phosphatase, beta-galactosidase, and acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride and phycoerythrin; an example of a luminescent material includes luminol; examples of bioluminescent materials include luciferin, and aequorin; and examples of suitable radioactive material include iodine (¹²¹I, ¹²³I, ¹²⁵I, ¹³¹I), carbon (¹⁴C), sulfur (³⁵S), tritium (³H), indium (¹¹¹In, ¹¹²In, ¹¹³mIn, ¹¹⁵mIn), technetium (⁹⁹Tc, ⁹⁹mTc), thallium (²⁰¹Ti), gallium (⁶⁸Ga, ⁶⁷Ga), palladium (¹⁰³Pd), molybdenum (⁹⁹Mo), xenon (¹³³Xe), fluorine (¹⁸F), ¹⁵³Sm, ¹⁷⁷Lu, ¹⁵⁹Gd, ¹⁴⁹Pm, ¹⁴⁰La, ¹⁷⁵Yb, ¹⁶⁶Ho, ⁹⁰Y, ⁴⁷Sc, ¹⁸⁶Re, ¹⁸⁸Re, ¹⁴²Pr, ¹⁰⁵Rh, and ⁹⁷Ru. Techniques for conjugating such moieties to antibodies are well-known.

Once an antibody or functional part thereof has been produced, it may be purified by any method known in the art for purification of an immunoglobulin molecule, for example, by chromatography (e.g., ion exchange, affinity, particularly by affinity for the specific antigens Protein A or Protein G, and sizing column chromatography), centrifugation, differential solubility, or by any other standard technique for the purification of proteins. In addition, the antibodies of the present disclosure or fragments thereof can be fused to heterologous polypeptide sequences to facilitate purification or detection.

The invention also provides a kit including one or more antibodies of the present disclosure. The kit may include one or more containers filled with one or more of the antibodies of the invention. Additionally, the kit may include other reagents such as buffers and solutions needed to practice the invention are also included. Optionally associated with such container(s) can be a notice or printed instructions. A kit can include packaging material. As used herein, the phrase “packaging material” refers to one or more physical structures used to house the contents of the kit. The packaging material is constructed by well-known methods, preferably to provide a sterile, contaminant-free environment.

Also included in the present disclosure are compositions including one or more of the antibodies described herein. A composition may also include, for example, buffering agents to help to maintain the pH in an acceptable range or preservatives to retard microbial growth. A composition may include, for example, carriers, excipients, stabilizers, chelators, salts, or antimicrobial agents. As used herein, a composition is not a polyclonal antiserum.

Also included in the present disclosure are hybridoma cell lines, transformed B cell lines, and host cells that produce the monoclonal antibodies of the present disclosure; the progeny or derivatives of these hybridomas, transformed B cell lines, and host cells; and equivalent or similar hybridomas, transformed B cell lines, and host cells. Progeny or derivatives thereof may produce an antibody with one or more of the identifying characteristics, such as, for example, isotype and antigen specificity, of the antibody produced by the parental line.

The present disclosure includes assay methods for the identification of agents that inhibit the aggregation of proteins, including, but not limited to, agents that inhibit the aggregation of proteins in neural cells and agents that inhibit the aggregation of proteins in the vestibular ganglia.

Such methods may include culturing in vitro appropriate neural cells expressing a genetic variant of the human PCDHGA10 gene as described herein, a genetic variant with a premature stop codon within intracellular/cytoplasmic domain of the short isoform the human PCDHGA10 polypeptide. Such cells include human neural progenitor cells, such as for example ATCC® ACS-5005™ human neural progenitor cells.

Such cells may be transfected to express a genetic variant with a premature stop codon within intracellular/cytoplasmic domain of the short isoform the human PCDHGA10 polypeptide, including, but not limited, wherein the genetic variant is NM_032090.1:c.2476_2477dup in the protocadherin gamma A10 gene (PCDHGA10); NM_032090.1:c.2477dup in the protocadherin gamma A10 gene (PCDHGA10); NM_032090.1:c.2467_2468insA (p.Lys823_Lys824fs) in the protocadherin gamma A10 gene (PCDHGA10); or the SNP allele Rs113784532, rs369101565, rs752029921, or rs750612188.

Such cells may be contacted with the agent and protein aggregation within the cells and/or cell survival determined by any of a variety of methods, including, but not limited to, any of those described in the examples included herewith. In some embodiments, protein aggregation in the cells may be assayed with one or more of the antibodies described above.

The present disclosure is illustrated by the following examples. It is to be understood that the particular examples, materials, amounts, and procedures are to be interpreted broadly in accordance with the scope and spirit of the invention as set forth herein.

### EXAMPLES

### Example 1

### Identification of a Genetic Mutation Underlying Familial Cases of Recurrent Benign Paroxysmal Positional Vertigo

BPPV is the most common cause of vertigo in humans, but the molecular etiology is completely unknown. Evidence suggests that genetic factors may play an important role in some cases of BPPV, particularly in familial cases, but the responsible gene variations/mutations have not been identified. With this example, whole exome sequencing (WES), including sequencing of untranslated regions (UTR), was used in familial cases of benign paroxysmal positional vertigo (BPPV) to search for candidate variants that may be responsible for heightened susceptibility to recurrent BPPV. Whole exome sequencing, including sequencing of untranslated regions, was performed in 12 families and Sanger sequencing of an additional 29 families (n=xx affected, unaffected) with recurrent BPPV in non-Hispanic whites from the US Midwest region, to identify the genetic variations/mutations responsible for heightened susceptibility to BPPV. In silico and experimental analyses of candidate variants show that an insertion mutation rs113784532 (frameshift causing truncation) in the neural cadherin gene PCDHGA10 (protocadherin-gamma 10) is an exceedingly strong candidate (p=1.1×10-4 vs. sample controls; p=1.5×10-100 vs. ExAC data). In mouse inner ear tissues, the expression of Pcdhga10 is only in the ganglia and increases with age. The mutant protein forms aggregates in the vestibular ganglia of BPPV patients, which also worsens with age. The data suggest that the mutation accelerates vestibular ganglia degeneration, which is a novel finding in BPPV etiology.

### Materials and Methods

Human subjects. This study was approved by the Institutional Review Board (IRB) at Boys Town National Research Hospital (BTNRH) (approval number 15-01-F) in accordance with institutional, federal, and international guidelines. Study invitation letters were sent to patients diagnosed with BPPV at the BTNRH Vestibular Clinic between 2009-2015 and potential control participants. Familial cases were identified either through our medical records, or through participants. Participants were asked to invite their affected (any age) and unaffected blood relatives (older than 50) to participate in the study. The affected relatives were confirmed to be diagnosed with BPPV by a specialist at BTNRH or elsewhere.

The study goal, procedure, risks, and benefits were explained to individuals who responded to the study invitation before signing the informed consent and HIPAA authorization forms. Subjects were then asked to collect their saliva samples using the ORAGENE™ Self-Collection Kits (Cat #OGR-500, DNA Genotek Inc., Ottawa, ON, Canada) and complete a detailed questionnaire. The questionnaire asks about demographic information (name, gender, age, weight, height, race and whether live in rural or urban area), BPPV related information (timeframe and location of diagnosis, method of treatment, recurrence, family history, season of onset, incidents or conditions prior to BPPV onset and comorbidities accompanying BPPV symptoms), medications, medical history, diet, family history of hearing loss/deafness and other hearing problems, and whether participants had experienced natural or surgical menopause (hysterectomy/oophorectomy) in women and prostate removal surgery in men.

The inclusion criteria for the enrollment of familial BPPV cases were: (1) besides the participant, at least two blood relatives (live or deceased) in the family had been clinically diagnosed with BPPV; (2) unaffected blood relatives were aged 50 years or older; (3) the subject signed informed consent and HIPAA authorization forms; (4) the subject completed the questionnaire. Families with two or more available affected individuals were selected for WES, along with unaffected blood relatives as controls. While most of the selected participants had recurrent BPPV, recurrence was not a requirement, as long as they are familial cases. Sample sets had a female/male ratio of 2.1:1, which is consistent with previous reports of higher prevalence of BPPV in females (Ogun et al., 2014, Menopause; 21:886-889, Liu et al., 2017, Front Aging Neurosci; 9:404).

Inclusion criteria for the enrollment of independent controls were: (1) subjects were 50 years or older; (2) subjects never had BPPV; (3) subjects did not have hearing impairments or other hearing problems; (4) blood relatives did not have BPPV, hearing impairments or other hearing problems; (5) subjects signed informed consent and HIPAA authorization forms; (6) subjects completed the questionnaire.

Inclusion criteria for the enrollment of non-BPPV families were similar to those for the independent controls.

Exclusion criteria were head trauma, serious infection (especially toxoplasmosis, rubella, cytomegalovirus, Herpes simplex virus, meningitis), aminoglycoside exposure, significant low birth weight (<4 lb, if known), inner ear structural abnormalities (if known), hyperbilirubinemia, and autoimmune disease (diabetes, autoimmune thyroid diseases, multiple sclerosis, myasthenia gravis). If affected family members do not share the same comorbidity, then the condition was not used as an exclusion criterion.

### Genomic DNA Preparation

Saliva samples were collected from the participants using ORAGENE™ Self-Collection Kits (Cat #OGR500) and genomic DNA (gDNA) was extracted using the Oragene prepIT-L2P kit (Cat #PT-L2P-5, DNA Genotek Inc.) following the manufacturer's protocol. The quality and concentration of genomic DNA (gDNA) was evaluated by agarose gel electrophoresis and NanoDrop OneC spectrophotometer (Thermo Fisher Scientific, Wilmington, DE, USA).

### Whole Exome Sequencing (WES) and Bioinformatics Analysis

Twenty-eight BPPV affected and seven unaffected individuals were selected for WES including 5′ and 3′ UTRs. The age, sex and BPPV status are listed in Table 1. The unaffected individuals were over 57 years old, (and 20 years older than the onset age of the family member.

WES+UTR sequencing and initial bioinformatic analysis were performed by Otogenetics Corporation (Atlanta, GA, USA). Briefly, high molecular weight gDNA with good integrity and good optical density ratio at 260/280 (˜1.8) was fragmented using a Bioruptor sonicator (Diagenode, Inc., Denville, NJ, USA) to an average size of 300-500 bp. Fragmented gDNA was tested for size distribution and concentration using an Agilent Tapestation 2200 and Nanodrop. Illumina libraries were then made from qualified fragmented gDNA using SPRIworks HT Reagent Kit (Cat #B06938, Beckman Coulter, Inc. Indianapolis, IN, USA) and the resulting libraries were subjected to exome enrichment using SureSelectXT Human All Exon version 5-UTRs (Cat #5190-6215, Agilent Technologies, Wilmington, DE, USA) following manufacturer's instructions. Enriched libraries were tested by an Agilent Bioanalyzer 2100 and sequenced on an Illumina HiSeq2500 (Illumina, San Diego, CA, USA) which generated 106-bp paired-end reads which generated 106-bp paired-end reads with an average of 63× coverage. FASTQC (Babraham Institute, Cambridge, UK) was used to analyze the quality of raw data.

After removing adapter sequences and low-quality reads (for example, those with too many Ns or low quality bases), the clean data were mapped against the human reference genome (GRCh37/hg19) using BWA algorithm with default settings (Li and Durbin, 2010, Bioinformatics; 26(5):589-95). Duplicate reads were removed using Picard (available on the worldwide web at picard.sourceforge.net). The Genome Analysis Tool Kit (GATK-Lite) toolkit (v3.8) (McKenna et al., 2010, Genome Res; 20(9):1297-303) module IndelRealigner and BaseRecalibrator were used to preprocess the alignments. During base quality recalibration, dbSNP141 variants were used as known sites, according to GATK Best Practices recommendations (DePristo et al., 2011, Nat Genet; 43(5):491-8). Target-capture efficiency metrics were determined using Target region coverage calculator Version 0.0.1. The realigned and recalibrated BAM file was used as an input to UnifiedGenotyper module from the GATK-lite toolkit. Variant calls were restricted to the target regions (Agilent AV5 Human). Finally, variants were annotated by SnpEff v4.1 (Cingolani et al., 2012, Fly (Austin); 6(2):80-92), Polyphen, SnpSift (Cingolani et al., 2012, Front Genet; 3:35) and ClinVar (available on the worldwide web at ncbi.nlm.nih.gov/clinvar/) to retrieve information on the impact of each variant, predicted functional changes, 1000 Genome population allele frequency, and associated diseases, if applicable.

After removing low impact variants according to SnpEff/SnpSift, those meeting the following criteria were prioritized for Sanger sequencing of additional samples: (1) variants are shared by affected but not by unaffected family members; (2) variants have a minor allele frequency (MAF) of lower than 0.05, or a CADD (combined annotation dependent depletion) score above 9; (3) variants are shared by 2 or more families, known to be expressed in the inner ear according to Gene Expression Omnibus (GEO) or Pubmed, or known to affect balance/hearing according to Online Mendelian Inheritance in Man (OMIM) or Pubmed. Sanger sequencing of the candidate variants was also performed on the WES samples for confirmation. Because BPPV has a lifetime prevalence of 10% [von Brevern et al., J Vestib Res. 2015; 25(3-4):105-17], and not all cases have a genetic etiology, we used the criteria of MAF<0.05 assuming genetic heterogeneity (i.e. several genes are involved). Neither criterion for the MAF or CADD score was stringent, so as to minimize false negatives at this initial screening stage.

### Sanger Sequencing Validation

Sanger sequencing was carried out to confirm the variants identified by exome sequencing, and to determine whether the variants in potential causative genes co-segregated with the disease phenotype in additional BPPV families and independent controls which did not go through exome sequencing. Forward and reverse primers (listed in Table 3) were used to amply the regions containing the candidate variations. The PCR products were purified with a Wizard SV gel and PCR Clean-Up System (Cat #A9281, Promega, Madison, WI, USA) and sequenced on a 3730xl DNA Analyzer (Applied Biosystems, Foster City, CA, USA). For the validation of rs113784532 in PCDHGA10, PCR product was digested with the restriction enzyme BpmI (Cat #R0565S, NEB) and purified prior to Sanger sequencing. Nucleotide alterations were confirmed by visual inspection of the electropherograms displayed with Chromas 2.6.5 (Technelysium Pty Ltd, Australia).

### IHC in Celloidin-Embedded Human Temporal Bone Sections

Human temporal bone specimens were obtained within 12 hours (h) of death from subjects with or without BPPV history as previous described (Lopez et al., 2005, J Neurosci Methods; 145(1-2):37-46). The temporal bones were then stored in 10% neutral-buffered formalin at 4° C. for 1-3 weeks, decalcified with EDTA (0.27-0.48 M, with or without 1% formalin, pH 7.2-7.3) for 9 months, and dehydrated in graded ascending ethylic alcohol and embedded in celloidin over a 3-month period. Celloidin-embedded temporal bones were horizontally sectioned at 20 μm using a Leica cryostat (CM1850) and mounted in Super frost plus slides (Fisher Scientific). Sections were allowed to dry at room temperature for 4 hours and then stored at −80° C. until use.

To remove the celloidin, sections were placed in a glass Petri dish and immersed in 100% acetone for 2×15 min, then sequentially immersed in a mixture of sodium-ethoxide-100% ethanol (1:3) for 10 min; 100% ethanol, 50% ethanol, and distilled water (5 min each); hydrogen peroxide 3% in methanol (10 min), then rinsed with double distilled water. Slides were then placed horizontally in a glass Petri dish containing antigen retrieval solution (Vector Antigen Unmasking Solution, Vector Labs, Burlingame CA diluted 1:20 with distilled water) and heated in the microwave oven using intermittent heating of two 2-min cycles with an interval of 1 min between the heating cycles, allowed to cool for 15 min, followed by 10 min wash with PBS.

Sections were blocked in PBS containing 5% normal goat serum (Vector Labs, Burlingame, CA) and 0.5% Triton X-100 (Sigma) for 30 min, and incubated in rabbit-derived polyclonal anti-PCDHGA10 (1:50 in blocking buffer) (catalog #orb1035, Biorbyt, Cambridge, UK) for 48 hat 4° C. in a humid chamber. Specificity of this antibody was confirmed by Western blotting as described on the stated supplier's website. After three washes in PBS, Alexa-488 conjugated goat anti-rabbit polyclonal IgG (Molecular Probes, Carlsbad, CA) was added at a dilution of 1:600 and incubated at RT for 1 h in the dark. Slides were mounted in Vectashield mounting media (Vector Labs, Burlingame, CA) containing DAPI and pictures were taken using a Zeiss Axio Observer Z1 inverted microscope equipped with an AxioCam MRm camera and with GFP, DsRed and DAPI filter sets.

Human Genomic DNA Extraction from Celloidin-Embedded Sections

Human genomic DNA was extracted from archival celloidin-embedded human temporal bone sections as described by Wackym et al. (Wackym et al., 1995, Am J Otol; 16(1):14-20) with slight modifications. Briefly, celloidin-embedded human temporal bone sections were rinsed in PBS to remove the coverslips, individual temporal bone tissues were then scraped into 1.5 ml Eppendorf tubes containing 1:1 volume of ether and 100% ethanol for 2×30 minutes. After centrifugation, supernatants were removed and the precipitates were washed in double distilled water, centrifuged, and saved (repeat three times). The precipitates were then washed twice with 100% ethanol, vacuum dried in a Labconco Centrivap Concentrator (Labconco Corp., Kansas City, MO) and digested with 0.5 mg/ml proteinase K in 500 μl of digesting buffer (10 mM Tris, pH 7.5, 50 mM EDTA, 150 mM NaCl, 1% SDS) overnight at 55° C. Afterwards human genomic DNA was isolated with standard phenol/chloroform extraction method and used for PCR amplification. Forward and reverse primers for human PCDHGA10 listed in Table 3 were used to amply the region containing rs113784532. PCR products were purified and submitted for Sanger sequencing.

### Mice

C57BL/6J (as B6) mice were purchased from the Jackson Laboratory (Bar Harbor, Maine, USA) and maintained in the vivarium at Boys Town National Research Hospital (BTNRH). All animal procedures were approved by the Institutional Animal Care and Use Committee (IACUC) at BTNRH in accordance with federal and international guidelines.

### Generation of Digoxigenin-Labeled RNA Probes

Mouse inner ear tissues were collected, total RNA was purified using the RNeasy Plus Mini kit (Cat #74134, Qiagen Inc., Valencia, CA, USA) combined with extra on-column DNase treatment with an RNase-free DNase set (Qiagen), and first-strand cDNA was generated using SUPERSCRIPT® VILO™ MasterMix (Invitrogen, Grand Island, NY) according to the manufacturers' instructions. The resultant product was used as PCR template to amplify the cDNA sequences for generating RNA probes for in situ hybridization. Primers 5′-ACACTCGAGCTGTGAGAAAAAAGATCC-3′ (SEQ ID NO: 7) and 5′-ACATCTAGAGAAACGCCAGTCAGTG-3′ (SEQ ID NO: 8) were used to amplify a 109 basepair (bp) sequence for detecting mouse Pcdhga10 long isoform, and primers 5′-ACACTCGAGCTGTGAGAAAAAAGATCC-3′ (SEQ ID NO: 7) and 5′-ACATCTAGATTTGGGCTCAAGCACAACG-3′ (SEQ ID NO: 8) were used to amplify a 143 bp sequence for detecting a predicted mouse Pcdhga10 short isoform at the equivalent position of human PCDHGA10.

Amplification products were purified using QIAquick PCR purification kit (Qiagen) and cloned into pGEM-T easy vector (Promega, Madison, WI). Positive clones were analyzed, sequenced to identify orientation and linearized with XhoI or XbaI. Digoxigenin (DIG)-labeled single-strand antisense and sense RNA probes for hybridization were generated using the DIG RNA Labeling Kit (SP6/T7) (Cat #11175025910, Roche Molecular Biochemicals, Alameda, CA) according to the manufacturer's instructions.

### Tissue Preparation for Fluorescence In Situ Hybridization

Animals were deeply anesthetized with ketamine-xylazine (ketamine: 200 mg/kg; xylazine: mg/kg body weight) and then decapitated. All following steps were carried out under RNase-free conditions with DEPC-treated solutions or buffers diluted by DEPC-treated water. inner ears were dissected in PBS and fixed in 4% PFA in PBS for 4 hours at room temperature (RT), decalcified in M EDTA (pH7.4) overnight, dehydrated in 30% sucrose prepared in PBS, embedded in O.C.T. compound at below −20° C., sectioned at 9 μm using a MICROM HM-505 N cryostat (Microm, Germany), thaw-mounted on Superfrost Plus glass slides (Thermo Fisher Scientific) and air-dried. The sections were stored at −80° C. until processed for fluorescent in situ hybridization (FISH).

### Fluorescent In Situ Hybridization

Frozen tissue sections were warmed to room temperature (RT) and desiccated for 20 min at post-fixed in 4% PFA, followed by two washes in PBS. Then sections were treated with 5 μg/ml proteinase K (Thermo Fisher Scientific) in 50 mM Tris buffer, pH 8.0, containing 5 mM EDTA, for 5 min at 37° C. After two washes in 2×SSC, sections were acetylated with 0.1 M triethanolamine, pH 8.0, containing 0.25% (v/v %) acetic anhydride for 20 min, and rinsed in 2×SSC. Pre-hybridize sections with pre-warmed pre-Hyb solution composed of 50% formamide, 4×SSC, 10% Dextran sulfate, 1× Dernhardt's and 50 μg/ml yeast tRNA (Sigma-Aldrich, St. Louis, MO, USA) in an RNase free humid chamber containing thin layer of 3M Whatman paper soaked with Chamber Mix (4×SSC/50% formamide) for 2 h at 55° C. Then replace pre-Hyb solution with 100 μl/slide Hybridization buffer (for 1 ml pre-warmed pre-Hyb, add 15 μl 20 mg/ml sheared, denatured herring sperm DNA and 0.1-0.5 μg/ml DIG-labeled anti-sense/sense RNA probe), cover slides with cover slips and incubate overnight at 55° C. in the humid chamber.

Following hybridization, sections were rinsed in 2×SSC to wash-off the cover slips and treated with 20 μg/m1RNase A (Sigma-Aldrich) in 10 mM Tris, pH 8.0, containing 500 mM NaCl and 1 mM EDTA, for 30 min at 37° C., followed by additional stringency washes in 2×SSC/50% formamide (5 min), 1×SSC (5 min), 0.5×SSC (5 min), at 50° C. and three washes in PBS at RT. After that, sections were stained using the ALEXA FLUOR™ 488 Tyramide SUPERBOOST™ Kit (Cat #B40922, Thermo Fisher Scientific) combined with rabbit-derived monoclonal Digoxigenin antibody (Cat #700772, Thermo Fisher Scientific) according to the manufacturer's instructions. Briefly, sections were blocked with 10% normal goat serum for 60 min at RT and incubated with Digoxigenin antibody (1: 500 in blocking buffer) overnight at 4° C., followed by three washes in PBS. Sections were then incubated with poly-HRP-conjugated goat anti-rabbit secondary antibody (1: 600 in blocking buffer), together with DAPI (Sigma-Aldrich, St. Louis, MO, USA) at a dilution of 1:10,000, in the dark for 60 min at RT. After three washes in PBS, for the signal enhancement, the tyramide working solution was applied to the sections for 6 min at RT, and the stop solution was used to terminate the HRP reaction. After three more washes in PBS, sections were mounted in Fluoromount-G and fluorescence images were acquired using a Zeiss Axio Observer Z1 inverted microscope equipped with an AxioCam MRm camera and with GFP, DsRed and DAPI filter sets.

### Results

### Characteristics of Families

Families in this study all had recurrent BPPV. For WES (+UTR) analysis presented below, 2-3 affected and 0-2 of unaffected blood relatives from 12 BPPV families of non-Hispanic white from the US Midwest were chosen on the basis of pedigree analysis and the availability of DNA (saliva) samples. Since BPPV has a reported mean onset age of 49.4 years (Von Brevern et al., 2007, J Neurol Neurosurg Psychiatry; 78(7):710-5; and Ogun et al., 2014, Menopause; 21:886-889), all the selected unaffected family members were at least 20 years older than the oldest onset age in the family at the time of sample collection. In the samples, the mean onset age was also 47, and the mean age of the unaffecteds was 67. In total, 28 affecteds, 7 unaffected relatives and 7 independent controls were selected for WES (see Table 1).

### Mutation Detection

On average, each exome had 97.6% covered at >10×, and 93.0% at >20×, with an average coverage of 63-fold. Approximately 180,000-200,000 single nucleotide variants (SNVs) and insertion-deletions (indels) per exome were detected. A tiered approach was used for variant selection, as depicted in FIG. 2. After variants' effects on protein functions were annotated by using SnpEff 4.1, PolyPhen, SIFT and ClinVar, variants with high impact or clinical significance were selected, resulting in a total of 800-1000 variants per exome. As genetic heterogeneity was anticipated, each family was first considered separately and selected the variants that were shared among all affected individuals in the family but not among unaffected members (including differences in homozygous and heterozygous state). This resulted in 13-227 variants per family. Next, variants with minor allele frequencies (MAF) of <0.05 or with Combined Annotation Dependent Depletion (CADD) scores of >9 were selected, which narrowed down the list to a total of 85 variants for the entire sample set. By comparing all families together, variants with contradicting status among affecteds and unaffecteds in different families were excluded, resulting in a total of 22 selected variants from all families. Finally, variants that were shared by 2 or more families, or were within genes known to be expressed in the inner ear according to Gene Expression Omnibus (GEO) and Pubmed or known to affect balance/hearing according to Online Mendelian Inheritance in Man (OMIM) and Pubmed, were given higher priority for follow-up. A total of 18 variants (Table 2) remained at this stage. Sanger sequencing of the 18 variants in additional 18-30 BPPV families resulted in an exceedingly strong candidate variant, plus at least a second promising variant (which is described in more detail in Example 2). Sanger sequencing of the 18 variants was also performed on the WES samples, which confirmed the WES results. In all, a total of 42 BPPV families and 50 non-BPPV families were analyzed, and one proband from each family was used for allele frequency calculations.

The strongest candidate is a heterozygous insertion mutation rs113784532 (NM_032090.1: c.2477dup, previously known as rs369101565/rs752029921/rs750612188), in the gene protocadherin gamma A10 (PCDHGA10) (FIG. 3A). This frameshift mutation causes a premature stop, truncating the PCDHGA10 short isoform (FIG. 3B). According to dbSNP152, the MAF of this mutation is 0.0002, yet Sanger sequencing revealed that 19 out of 61 affected familial BPPV cases in our sample set had this mutation (p=1.36×10⁻¹¹⁷). Among the positive families, this variant co-segregated with BPPV-affected individuals. This variant also tends to make the onset age of recurrent BPPV earlier, with an average onset age of 44.0 years old among those with the variant as compared with 54.4 years of onset age among those without (p=0.054).

### Mutant PCDHGA10 Protein Forms Excessive Intracellular Aggregates in the Vestibular Ganglia of BPPV Patients

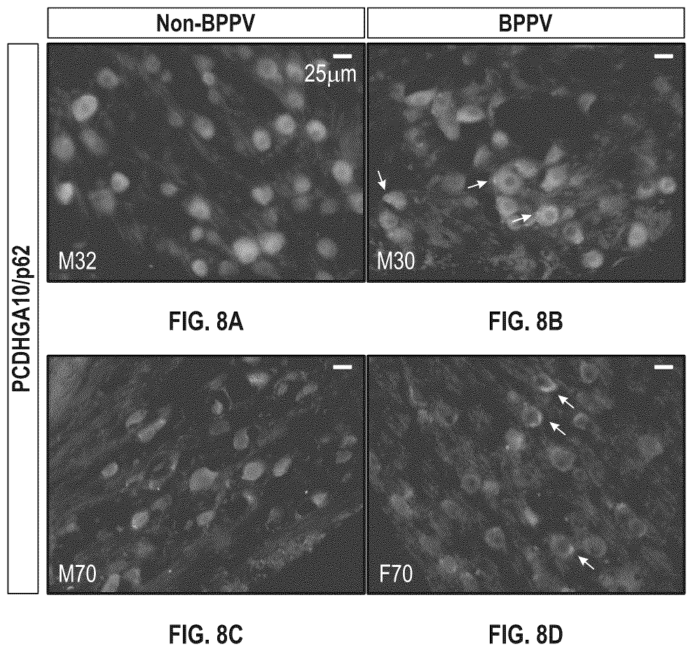

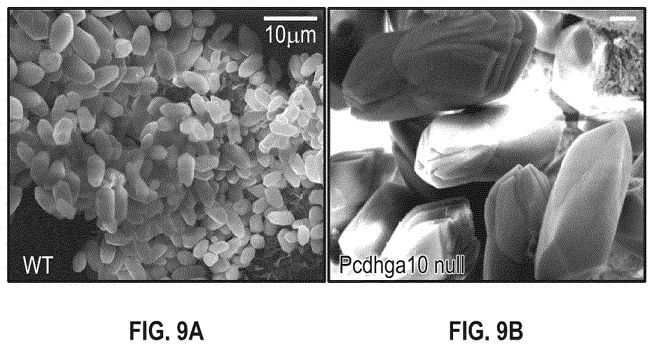

Fluorescent immunostaining was carried out to examine the expression of PCDHGA10 in the vestibular ganglia of human temporal bone. FIGS. 3D and 8D show that mutant PCDHGA10 formed large intracellular aggregates that appear to be localized in macroautophagy in the vestibular ganglia of both young (30-year-old) and old (70-year-old) BPPV patients. However, in the non-BPPV vestibular ganglia at similar ages, the protein expression was evenly distributed in the cytosol (FIGS. 3C, 8A, and 8C).

Expression Pattern of Long and Short Pcdhga10 mRNA in Mouse Vestibular Ganglia Revealed by Fluorescence In Situ Hybridization Assay

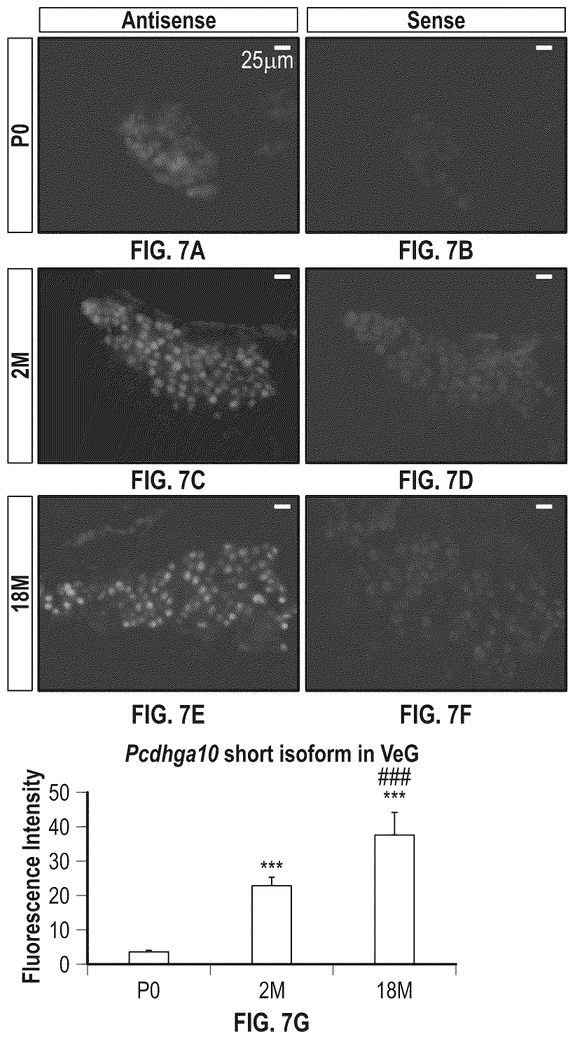

To investigate which isoform of PCDHGA10 might be involved in aggregates formation in the vestibular ganglia, the distribution of long isoform of mouse Pcdhga10 mRNA was investigated, as well as the predicted short isoform that is equivalent to that of human PCDHGA10, at different ages using sensitive fluorescence in situ hybridization (FISH). The results are shown in FIG. 7 and indicate that in the vestibular ganglia of postnatal day 0 (P0), 1-month-old (1M), 2-month-old (2M) mice, the expression levels of mPcdhga10 long isoform are extremely low (FIGS. 7A, 7C, and 7E), whereas the fluorescent signal of long isoform antisense probe was stronger in the 18-month-old (18M) mouse vestibular ganglia (FIG. 7G). In contrast, the expression of mPcdhga10 short isoform were already detectable, although not very high, at both P0 and 1M (FIGS. 8A and 8C), and the fluorescent signals were even much stronger in the vestibular ganglia of 2M and 18M mice (FIGS. 8E and 8G). The specificity of all the in situ hybridizations was controlled on adjacent sections under same condition with the corresponding sense probes, no hybridizations were seen, indicating that no nonspecific binding to RNA or DNA occurred. These data demonstrated that, in mouse vestibular ganglia, the expression level of Pcdhga10 short isoform was much higher than the long isoform at all tested age groups, suggesting that the short isoform is the predominant Pcdhga10 in vestibular ganglia.

### Discussion

This example is the first study to identify a genetic mutation causing or exacerbating idiopathic BPPV in humans. Given the high prevalence and debilitating nature of recurrent BPPV, often dually inflicted by age-related hearing loss, this type of genetic study is overdue. The whole exome plus UTR approach allowed simultaneous identification of multiple candidate genes, yielding efficient and speedy discovery of the genetic causes of recurrent BPPV.

This example shows that a rare variant in the short isoform of the PCDHGA10 gene is strongly associated with familial cases of recurrent BPPV. PCDHGA10 belongs to the γ-subfamily of the protocadherins (PCDH) super family of 70 genes. PCDHs are neuronal adhesion molecules that are concentrated in neurons and synapses. Fifty-eight of the PCDHs are tandemly arrayed in three clusters (α, β, and γ) on human chromosome 5 and mouse chromosome 18. Each cluster contains multiple “variable exons” encoding the extracellular, transmembrane, and cytoplasmic domains of individual isoforms. Within the Pcdh-α and γ clusters, each “variable exon” is transcribed from its own promoter and spliced to constant exons that encode a shared C-terminal constant domain (Obata et al., 1998, Cell Adhes Commun; 6:323-333; Sugino et al., 2000, Genomics; 63:75-87; Wang et al., 2002, Genes Dev; 16:1890-1905; and Tasic et al., 2002, Mol Cell; much like immunoglobulin (Ig) and T cell receptor (TCR) gene clusters. Such diversity and genomic rearrangement, coupled with evidence that different neurons express different PCDHs (Kohmura et al., 1998, Neuron; 20(6):1137-51; Frank et al., 2005, Mol Cell Neurosci; 29(4):603-16; Wang et al., 2002, Genes Dev; 16:1890-1905; Wang et al., 2002, Neuron; 36:843-854; Phillips et al., 2003, J Neurosci; 23:5096-5104; Blank et al., 2004, Mol Cell Neurosci; 26(4):530-43; and Esumi et al., 2005, Nat Genet; 37(2):171-6), suggest that defined sets of PCDH expression may engage in the establishment of specific neuronal connections.

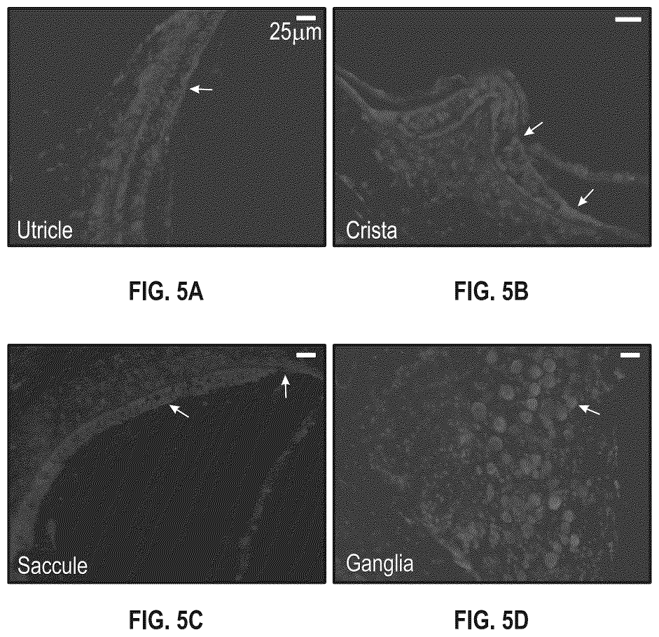

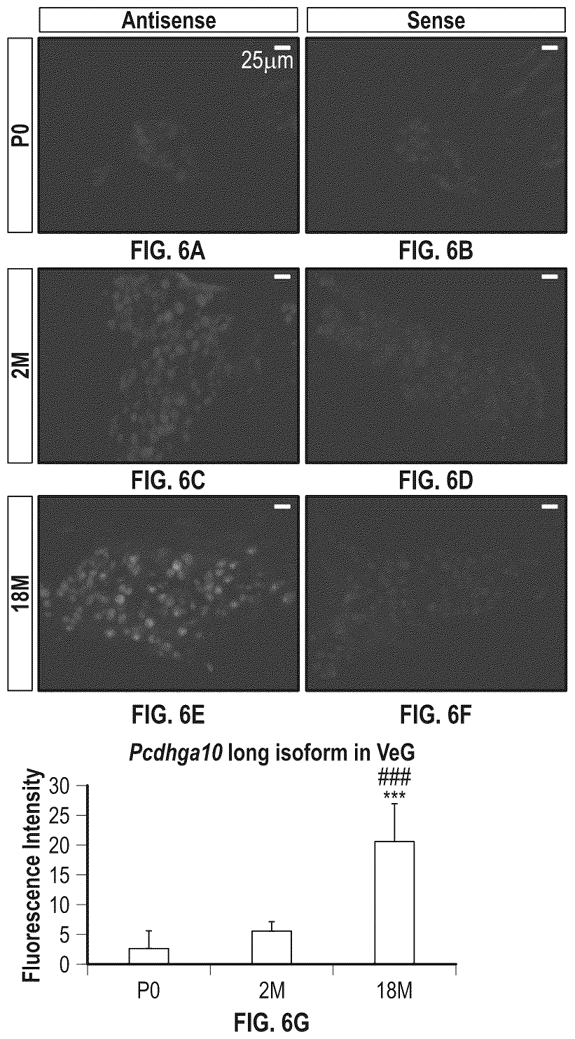

Unlike classical cadherins, which are present at the cell surface, γ-Pcdhs have a prominent intracellular presence in neurons, particularly in endolysosomes (Wang et al., 2002, Genes Dev; 16:1890-1905; Wang et al., 2002, Neuron; 36:843-854; Phillips et al., 2003, J Neurosci; 23:5096-5104; and Fernadez-Monreal et al., 2010, Eur J Neurosci; 32(6):921-31). The intracellular variable domains of γ-PCDHs are the most important contributor to localizing the protein to endolysosomes, but the constant domain is needed for 100% efficiency (O'Leary et al., 2011, Mol Blot Cell; 22:4362-4372; and Shonubi et al., 2015, BMC Cell Biol; 16:28). Fluorescent immunostaining results have shown that PCDHGA10 formed excessive intracellular aggregates in the vestibular ganglia of the BPPV patients, whereas in that of non-BPPV individuals the protein appeared to be low and evenly distributed in the cytosol (FIG. 5). Although one cannot ascertain whether the aggregates were comprised of the long or short isoform of PCDHGA10 due to the antibody we applied, the in situ hybridizations carried out on mouse inner ear sections revealed that, in vestibular ganglia, the expression levels of Pcdhga10 short isoform are notably higher than the long isoform throughout life (FIGS. 7 and 8). These data indicate that the short isoform is the predominant isoform in vestibular ganglia and probably essential for both neurogenesis and survival of vestibular ganglia. Functional disruptions of this gene, such as mutations at critical position, is likely causing malfunction of vestibular ganglia. The excessive PCDHGA10 aggregates we observed in the vestibular ganglia of BPPV patients (FIG. 5), therefore, are probably most, if not all, comprised of the mutant short isoform. The PCDHGA10 mutation detected with this example in the familial BPPV cases may aggravate the aggregate formation for two reasons: (1) The truncation may cause the protein to be less efficiently targeted to the autophagosomes-lysosomes for degradation, and/or (2) The truncation may not be fully digestible by lysosomal enzymes.

The current belief is that, when otoconia dislocate from the utricle into the semi-circular canals, or clots of CaCO₃ debris aggregate in the canals, the normal mechanics of the endolymph is altered and BPPV ensues. The identification of PCDHGA10 as strong candidate gene causing familial cases of recurrent BPPV shifts this paradigm in that neurosensory degeneration may also cause or at least exacerbate BPPV. This finding can be of benefit to the families from several aspects. Firstly, the information can be used for diagnosis and counseling. Results from genetic testing can provide a mental relief for the affected families because they feel that at least they know what is causing their intense vertigo. Secondly, there may be generic remedies available already. For example, if the gene mutation causes neurosensory degeneration, generic remedies, such as anti-cell death or neurotrophic medication, are already available to slow down functional deterioration of neurons and sensory cells; if imbalanced neurotransmission facilitates recurrent BPPV, there is a myriad of existing medications to modulate neurotransmission. Thirdly, depending on the severity and type of mutation in causing recurrent BPPV, gene therapies using small molecules such as RNAi and oligos, or genome editing using CRISPR/Cas9 can be implemented on recurrent BPPV patients in the future.

### Example 2

### Caspase 10 (CASP10), a Second Genetic Mutation Associated with Familial Cases of Recurrent Benign Paroxysmal Positional Vertigo

Following the procedures described in more detail in Example 1, a second genetic variant associated with familial cases of recurrent Benign Paroxysmal Positional Vertigo (BPPV) has been identified. This variant is the heterozygous missense variant rs13010627 (NM_032974.5: c.1228G>A) in the apoptosis gene Caspase 10 (CASP10). This mutation results in a valine to isoleucine substitution at position 410 in CASP10 isoform 2. According to 1000G, the MAF of this variant is 0.0192 (ExAC 0.0426). In the sample set described in example 1, 11 out of 57 affecteds carried this heterozygous variant (p=1.14×10-8), whereas the unaffected family members were all homozygous for the wild-type allele (G/G) (FIG. 4).

This second strong candidate variant, c.1228G>A (V410I in protein), lies in Caspase-10 gene (CASP10, also known as MHC4 or FLICE2) located in human chromosome 2q33. The protein product CASP10 is a cysteine protease, and the variant is located in the p17 large subunit of the protease. Like its paralogue caspase-8, CASP10 is recognized as an initiator caspase in death receptor apoptotic pathway (Wang et al., 2001, Proc Natl Acad Sci USA; 98(24):13884-8). Pro-CASP10 is an inactive zymogen with a long N-terminal prodomain, containing two death effector domains (DEDs), followed by the catalytic domain which can be further processed into a large (p17) and a small subunit (p12). An apoptotic stimulus can trigger the formation of death-inducing signaling complex (DISC), in which the pro-CASP10 becomes mature enzyme with proteolytic activity via dimerization and subsequent auto-catalytic cleavage (Turk et al., 2002, Biol Chem; 383(7-8):1035-44; Turk and Stoka, 2007, FEBS Lett; 581(15):2761-7; Wachmann et al., 2010, Biochemistry; 49(38):8307-15; and Wang et al., 2001, Proc Natl Acad Sci USA; 98(24):13884-8). Once CASP10 is activated, it can cleave the downstream effector caspases (Caspase-3, -6 and -7) and initiate the execution phase of apoptosis. CASP10 is highly conserved throughout evolution but is lost in the rodent lineage (Janicke et al., 2006, Science; 312(5782):1874; Eckhart et al., 2008, Mol Blot Evol; 25(5):831-41; and Sakamaki et al., 2015, Bioessays; 37(7):767-76). Due to the absence of CASP10 in mice, the most commonly used animal model, the functional analysis of CASP10 is less amenable and mostly carried out in cultured cells.

The same V410I variant but in homozygous state, along with other variants (L285F, C401S, I406L, M147T and Q257stop) in CASP10 gene, has been found to be associated with human autoimmune lymphoproliferative syndrome (ALPS) (Wang et al., 1999, Cell; 98(1):47-58), non-Hodgkin lymphomas (NHL) (Shin et al., 2002, Blood; 99(11):4094-9) and gastric cancer (Park et al., 2002, Oncogene; 21(18):2919-25). The authors suggest that V410I may mildly reduce the apoptotic function of CASP10, thus leading to uncontrolled proliferation in these diseases. However, in a study by Zhu and coworkers (Zhu et al., 2006, Hum Genet; 119(3):284-94), there are more unaffected individuals than ALPS patients (2:1) carrying homozygous V410I. In addition, previous case-control studies indicate that the V410I missense variant of CASP10 is actually associated with reduced familial breast cancer risk in the general population (Frank et al., 2006, Carcinogenesis; 27(3):606-9). These data suggest that V410I alone is not sufficient to cause ALPS, or it can even enhance apoptosis. A recent study shows that, while CASP10 has redundant function as CASP8, CASP10 inhibits CASP8-mediated cell death and switches the cellular response to NFKB-mediate cell survival (Horn et al., 2017, Cell Rep; 19(4):785-797).

CASP10 may be involved in maintaining the balance between cell death and survival. Indeed, Lamy et al. found that CASP10 is critical for maintaining the proper balance between pro-survival and pro-death autophagic responses, and can inhibit autophagy via cleaving BCLAF1, a BCL2-interacting protein, in multiple myeloma cells (Lamy et al., 2013, Cancer Cell; 23(4):435-49). Later, Guo et al. found that application of CASP10 inhibitor can increase autophagic cell death in acute myeloid leukemia cells (Guo et al., 2016, Oncol Lett; 12(2):1623-1629). The function of the V410I variant in the vestibular and auditory systems is totally unknown. In vitro functional assessments reveal that the genetic polymorphisms L285F, C401S and I406L can inhibit Fas- and TRAIL-induced apoptosis in lymphocytes and dendritic cells (Wang et al., 1999, Cell; 98(1):47-58; and Zhu et al., 2006, Hum Genet; 119(3):284-94). M147T and Q257stop variants identified in gastric cancer patients can severely impair CASP10 mediated apoptosis, whereas the V410I mutation has less severe but still significant effect (Park et al., 2002, Oncogene; 21(18):2919-25).

The current belief is that, when otoconia dislocate from the utricle into the semi-circular canals, or clots of CaCO₃ debris aggregate in the canals, the normal mechanics of the endolymph is altered and BPPV ensues. The identification of CASP10 as strong candidate genes causing familial cases of recurrent BPPV shifts this paradigm in that neurosensory degeneration may also cause or at least exacerbate BPPV. This finding can be of benefit to the families from several aspects. Firstly, the information can be used for diagnosis and counseling. Results from genetic testing can provide a tremendous mental relief for the affected families because they feel that at least they know what is causing their intense vertigo. Secondly, there may be generic remedies available already. For example, if the gene mutation causes neurosensory degeneration, generic remedies, such as anti-cell death or neurotrophic medication, are already available to slow down functional deterioration of neurons and sensory cells; if imbalanced neurotransmission facilitates recurrent BPPV, there is a myriad of existing medications to modulate neurotransmission. Thirdly, depending on the severity and type of mutation in causing recurrent BPPV, gene therapies using small molecules such as RNAi and oligos, or genome editing using CRISPR/Cas9 can be implemented on recurrent BPPV patients in the future.

### Example 3

### Molecular and Cellular Events Contributing to BPPV

Vertigo and balance deficits involving the vestibule of the inner ear are common, disruptive to daily living, and can be debilitating especially in the elderly, yet the molecular etiology of these conditions in humans is completely unknown.

Example 1 identified a strong candidate gene, PCDHGA10, associated with benign paroxysmal positional vertigo (BPPV), a prevalent age-related condition. Example 1 also showed that the PCDHGA10 protein product forms aggregates in old (but not young) vestibular ganglia in both humans and mice. It is expected that a mutation in the unique region of the short isoform worsens aggregation of the long isoform in the vestibular ganglia during aging.

Progressive neurodegeneration has many manifestations. The severe cases include Alzheimer's, Parkinson's, Huntington's diseases, and amyotrophic lateral sclerosis (AD, PD, HD, ALS). These devastating diseases all have at least one thing in common: protein aggregation in the respective neurons as a result of impaired autophagy.

This example examines molecular and cellular events contributing to benign paroxysmal positional vertigo, a prevalent vestibular disorder, and compare with one of the well-studied protein aggregate diseases, Alzheimer's Disease. This example pertains to the following issues: (1) does the peripheral nervous system also have age-related protein aggregation, and if so, (2) what is the timeline of the aggregation?

This example will first determine the approximate onset age of PCDHGA10 protein aggregation in human and mouse vestibular ganglia, and its association with the proteostasis/macroautophagy system. These findings will be compared with the vestibular ganglia of deceased individuals with BPPV and Alzheimer's disease.

This example will then study the effects of the mutant human PCDHGA10 on protein aggregation and neuronal survival in human and mouse neuronal cells. Age-related protein aggregate diseases may be much more common than previously realized, as BPPV is highly prevalent with a lifetime prevalence of 10%. Despite being the most common age-related condition of the balance/vestibular system, the molecular causes of BPPV are completely unknown at present.

Given the recent data that show presence of sensorimotor deficits long before the onset of memory loss in AD patients (Albers et al., 2015, Alzheimers Dement; 11:70-98), establishing the earliest timeline is critical for identifying the optimal window of opportunity for treatment and/or mitigation of these types of diseases in the future. The main reason for various drugs to fail in testing of AD treatment could be that by the time memory loss is severe, it is too late.

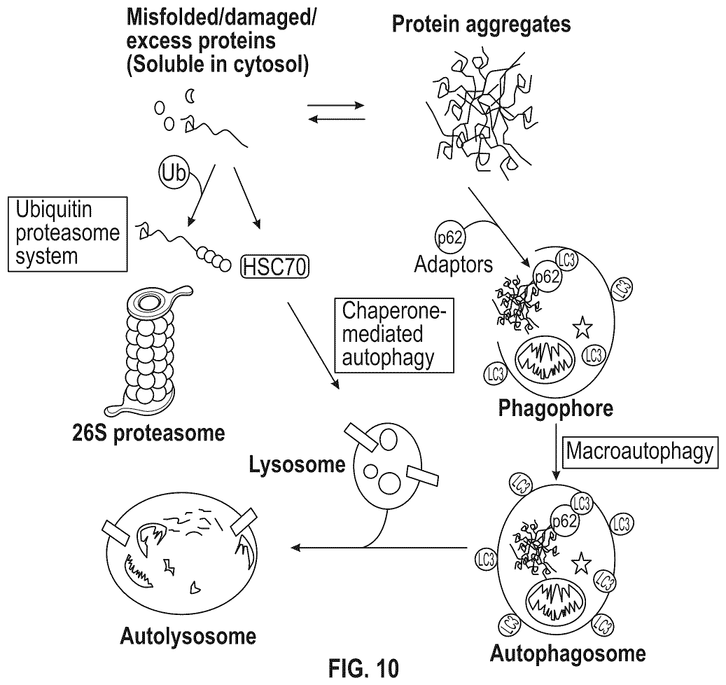

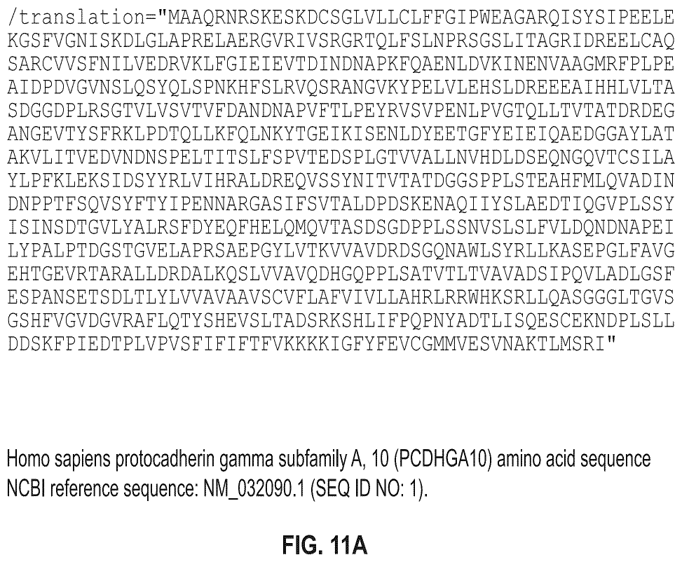

The elimination of dysfunctional, abnormal, or damaged cellular constituents, such as misfolded (or excess) proteins, aberrant protein aggregates, and malfunctioning organelles, is essential for neurons and other types of cells to maintain their proper function and to survive. Proteostasis is accomplished by two major routes: ubiquitin/proteasomal digestion (UPS) and autophagy (FIG. 10). Although autophagy has two types, chaperone-mediated and macroautophagy, the term “autophagy” usually refers to the latter. Insoluble protein aggregates and damaged organelles are eliminated by macroautophagy. Selective autophagic adaptors, such as p62/SQSTM-1, can target the protein aggregates into autophagosomes by interacting with LC3 (aka Atg8 in yeast). The autophagosomes are subsequently fused with lysosomes to form autolysosomes in which the aggregated proteins are degraded.

From yeast to man, autophagy is highly conserved. Under normal conditions, the activity of autophagy remains at basal level; but under adverse conditions, it is elevated for cell survival. Autophagy activity in many long-lived animal models is elevated, and inhibition of autophagy genes leads to cancellation of their longevity (Nakamura and Yoshimori, 2018, Mol Cells; 41:65-72). In contrast, autophagy impairment has been linked to the most severe neurodegenerative diseases in humans, such as Alzheimer's disease (AD), amyotrophic lateral sclerosis (ALS), Parkinson's disease (PD) and Huntington's disease (HD) (Metaxakis et al., 2018, Cells: 7(5):pii E37; and Menzies et al., 2017, Neuron; 93:1015-1034). These diseases affect millions of Americans: 5.7 million with AD (Alzheimer's Association, “2018 Alzheimer's disease facts and figures,” Alzheimers Dement; 14:367-429), over 20,000 with ALS (Mehta et al., 2018, MMWR Morb Mortal Wkly Rep; 67:1285-1289), nearly 1 million with PD (Marras et al., 2018, NPJ Parkinsons Dis; 4:21), and nearly 30,000 with HD (National Institute of Neurological Disorders and Stroke. Huntington's Disease Information Page, 2015).

The study of the human genetics of benign paroxysmal positional vertigo (BPPV) described in more detail in Example 1 identified a strong candidate gene PCDHGA10 whose mutation accounts for 45% of the familial cases of BPPV studied. The protein product forms aggregates in aging but not young vestibular ganglia in both humans and mice. Because BPPV is the most common cause of vertigo in humans, with a lifetime prevalence of 10% (Von Brevern et al., 2015, J Vestib Res; 25:105-117), thus suggests that protein aggregates-caused neuronal degeneration may be even more prevalent than previously thought and may affect both peripheral and central nervous systems. During aging, vestibular deficits are highly prevalent and can be debilitating and disruptive to daily living (Handa et al., 2005, Braz J Otorhinolaryngol; 71:776-782; and Lopez-Escamez et al., 2005, Eur Arch Otorhinolaryngol; 262:507-511). Such balance deficits are further magnified in elderly persons with vision deficits and/or cerebellar dysfunction.

Clinically, BPPV is believed to be caused by otoconia dislocation from the utricle (which detects linear head motion) to the semicircular canals (which sense rotational movement), although visualization of such events has not been possible. Otoconia are extracellular bio-crystals overlaying the sensory epithelium of the utricle and saccule in the inner ear and are composed of proteins and calcium carbonate. The crystals provide inertia mass that yield shearing force on the stereocilia of the sensory hair cells during head movement. While one third of BPPV cases in young people can be attributed to head trauma/injury, idiopathic BPPV cases are much more common in middle-aged and older people, which also tend to be recurrent (Cohen et al., 2004, J Otorhinolaryngol Relat Spec; 66:11-15; and Ogun et al., 2014, PLoS One; 9:e105546). In all, approximately 60% of BPPV cases are idiopathic (Katsarkas, 1999, Acta Otolaryngol; 119:745-749). Currently, no medication is available to treat or prevent BPPV. Therefore, the development of effective treatment for the recurrent cases is needed and is dependent on research on the molecular etiology of BPPV.

Otoconial and neural degeneration may cause or exacerbate BPPV given the increase of BPPV cases with age and the degenerative otoconia (Anniko et al., 1984, Acta Otolaryngol; 97:283-289; Igarashi et al., 1993, Acta Otolaryngol Suppl; 504:26-29; Ogun et al., 2014, Menopause; 21:886-889; and Ross and Pote, 1984, Philos Trans R Soc Lond B Blot Sci; 304:445-452) and 50% ganglia loss (Gacek, 2003, Ann Otol Rhinol Laryngol; 112:574-582) observed in the postmortem vestibules of BPPV patients. In fish, vestibular neuroectomy causes abnormal otoliths (otoconia are called otolith in fish) (Anken et al., 2000, Neuroreport; 11:2981-2983). Therefore, the ganglia loss may lead to otoconia degeneration, imbalanced neurotransmission, loss of contralateral inhibition, or as Gacek postulated, loss of inhibition by the otolithic organ on the crista in the canals. Taken together, the work of this example will provide mechanistic insight into the cellular and molecular etiology of BPPV. Such information will be particularly useful given the rapid advancement of gene therapy methods, such as using small molecules like RNAi and oligos, which have already been approved by the FDA in treating other diseases, and genome editing using CRISPR/Cas9, which is expected to be approved soon.

The proposed work of this example is among the first to detect and study age-related protein aggregation in the peripheral sensory nervous system. Because people with AD show sensorimotor deficits first, 5-15 years before the onset of cognitive decline (Albers et al., 2015, Alzheimer s Dement; 11:70-98), establishing the earliest timeline in the proposed work is critical for identifying the optimal window of opportunity for treatment and/or mitigation of these types of diseases in the future. The main reason for various drugs to fail in AD treatment testing could be that by the time memory loss is severe, it is too late. Earlier treatment/prevention may prove to be much more effective.

The hypothesis of neuronal degeneration as a contributing factor to BPPV etiology is another innovative aspect of the findings. Current medical imaging is not advanced enough to visualize whether BPPV is indeed caused by otoconia dislocation. In our genetic study of familial BPPV cases, none of the known otoconial genes appeared as a candidate in our preliminary study. Rather, the strongest candidate gene, PCDHGA10, is predominantly expressed in the ganglia of the inner ear and at a level that increases with age. This suggests that neuronal degeneration may contribute to BPPV or can lead to otoconia degeneration and subsequent dislocation. Somehow, the topic of vestibular ganglia aging has been neglected, likely due to few researchers working in this field and the difficult funding situation. The utilization of whole exome sequencing is novel in the vestibular field.

As described in Example 1, whole exome sequencing (WES+UTR) of familial cases of BPPV was performed using 12 families of non-Hispanic white from the US Midwest, each with 2-3 affecteds and 0-2 of unaffected blood relatives for a total of 28 affecteds and 14 unaffecteds. Each exome had at least 97.6% covered at >10×, and 93.0% at >20×. After removal of synonymous and low impact intronic variants, those meeting the following criteria were selected for Sanger sequencing of additional samples: (1) variants are shared by affected but not by unaffected family members, and variants versus phenotypes are consistent between affecteds and unaffecteds within a family and among different families; (2) variants have a minor allele frequency of lower than 0.05, or a CADD (combined annotation dependent depletion, a tool for scoring the deleteriousness of variants in the human genome) score above 9; (3) variants are shared by 2 or more families, known to be expressed in the inner ear (GEO, Pubmed) or known to affect balance/hearing (OMIM, Pubmed). Using this tiered selection process, a total of 18 variants were selected for Sanger sequencing of additional 18-29 BPPV families. Sanger sequencing of the 18 variants was also performed on the WES samples for verification. The strongest candidate is a heterozygous insertion mutation rs113784532 [NM_032090.1:c.2467_2468insA (p.Lys823_Lys824fs)] (MAF=0.0002 according to dbSNP), in the gene PCDHGA10 (protocadherin gamma A10 gene (Table 1). This frameshift mutation causes a premature stop, truncating the PCDHGA10 short isoform (FIG. 2).

Another promising candidate variant, described in more detail in example 2, is a heterozygous missense variant rs13010627 (NM_001206524.1: p.Val343Ile/c.1027G>A) (MAF=0.028), in the apoptosis gene Caspase 10 gene (Table 1).

PCDHGA10 belongs to the γ-subfamily of the protocadherins (PCDH) super family of 70 genes. PCDHs are concentrated in neurons and synapses. Fifty-eight of the PCDHs are tandemly arrayed in three clusters (α, β, and γ,) on human chromosome 5 and mouse chromosome 18. Each cluster contains multiple “variable exons” encoding the extracellular, transmembrane, and cytoplasmic domains of individual isoforms. Within the PCDH-α and γ clusters, each “variable exon” is transcribed from its own promoter and spliced to constant exons that encode a shared C-terminal constant domain (Obata et al., 1998, Cell Adhes Commun; 6:323-333; Sugino et al., 2000, Genomics; 63:75-87; Tasic et al., 2002, Mol Cell; 10:21-33; and Wang et al., 2002, Genes Dev; 16:1890-1905), much like the clusters encoding immunoglobulins (Ig) and Tcell receptors. The constant domain of PCDHs is identical within the same cluster, and highly conserved between clusters. Depending on the family member and interacting molecules, γ-PCDHs are important for synaptic formation and maturation, spatial patterning of axons and dendrites, dendritic pruning and self-avoidance, and neuronal survival (Garrett and Weiner, 2009, J Neurosci; 29:11723-11731; Kostadinov and Sanes, 2015, Elife; 4 (DOI: 10.7554/eLife.08964); and Molumby et al., 2017, Cell Rep; 18:2702-2714).

Unlike classical cadherins, which are present at the cell surface, γ-PCDHs have a prominent intracellular presence in neurons, particularly in endolysosomes (Wang et al., 2002, Neuron; 36:843-854; Phillips et al., 2003, J Neurosci; 23:5096-5104; and Fernandez-Monreal et al., 2010, Eur J Neurosci; 32:921-931). The variable domains of γ-PCDHs are the most important contributor to localizing the protein to endolysosomes (O'Leary et al., 2011, Mol Blot Cell; 22:4362-4372; and Shonubi et al., 2015, BMC Cell Biol; 16:28). There are not many studies on PCDHs, and most are focused on their roles in development. Findings in mutant mice suggest that the predominant role of γ-PCDHs is neuronal survival (Wang et al., 2002, Neuron; 36:843-854). However, little is known about their roles in adulthood, and details regarding their function in neurons are lacking. Nothing is known about that of PCDHGA10. The work of this example will begin to uncover the role of PCDHGA10 in the survival of the vestibular ganglia.

Fluorescent immunostaining (as described in more detail in Example 1) shows that PCDHGA10 expression increases with age in both human and mouse inner ears, coinciding with the mostly middle-age onset of idiopathic BPPV. In young adults, the protein expression is low and show only minute (if any) powder-like distribution in the cytosol. However, in the aging vestibular ganglia, PCDHGA10 forms large aggregates that appear to be localized in macroautophagy. Indeed, the protein co-localizes with p62, a macroautophagy protein. The antibody used (Cat #orb1025, Biorbyt) cannot differentiate the long and short forms of PCDHGA10.

Interestingly, based on microarray data in NCBI GEO database, PCDHGA10 expression (mRNA) is drastically increased in some cases (but not all) of the following (parentheses indicate tissues studied): (1) those with severe Alzheimer's disease (hippocampal CA1) (NCBI GEO database. PCDHGA10 expression in Alzheimer's disease at various stages of severity. 2018) or with neurofibrillary tangles (entorhinal cortex) (NCBI GEO database. PCDHGA10 expression in Alzheimer's disease: neurofibrillary tangles. 2018), (2) those under chronic stress (peripheral blood monocytes) (NCBI GEO database. PCDHGA10 expression in Chronic stress effect on peripheral blood monocytes. 2018), and (3) those with schizophrenia (cerebellar cortex) (NCBI GEO database. PCDHGA10 expression in Cerebellar cortex in schizophrenia. 2018). It is unclear whether the elevated expression of PCDHGA10 involves the long, short, or mutant isoform. The expression levels are low in the nervous tissues mentioned above, but it could be caused by the use of postmortem tissues.

To ensure scientific rigor, reproducibility, transparency, and to minimize bias, this example will employ complementary methods and analyses, and strictly apply appropriate, well-vetted scientific methods, and use independent tools to verify the results. For example, the WES and immunostaining studies described in Example 1 were done in a double blind way, and Sanger sequencing was used to verify the WES data in all cases. Blinding procedures will be employed with studies of postmortem human tissues. Further, both in vivo and in vitro methods will be employed and each experiment is confirmed by another independent method. For example, results from protein expression using quantitative fluorescent immunostaining will be confirmed by quantitative Western blotting and/or by gene expression using quantitative RT-PCR.

In animal studies, age-matched male and female mice will be used. With postmortem human samples, the approximate age group will be matched, and both males and females will be included. Missing data resulting from attrition or exclusion will be documented, and results (negative and positive) will be reported. To further ensure full transparency, de-identified genotypes and phenotypes will be deposited in a public database (dbGaP) after publication of the data, which also satisfies the resource-sharing requirement. In statistical analysis, all experiments will be designed to ensure that power will be high enough to detect departures from the null hypothesis. In power analysis, the results from preliminary and published studies will be used to guide the selection of sample sizes. Justification for number of samples is provided in each aim.

Briefly, Specific Aim 1 of this example will determine the approximate onset age of PCDHGA10 protein aggregation in human and mouse vestibular ganglia, and its association with the proteostasis/macroautophagy system.

The quantity of RNA expression versus protein accumulation of PCDHGA10 in mouse and human ganglia in young, middle, and old ages in both sexes will be compared. Samples with BPPV and Alzheimer's disease will be included in the old age group for comparison. All the postmortem human tissues and some of the mouse tissues needed for the study have been collected.

In vivo PCDHGA10 protein aggregation will be associated with autophagosomes-lysosomes (cellular disposal machinery of protein aggregates and damaged organelles) in the above human and mouse tissues.

Further, Specific Aim 2 of this example will determine if the PCDHGA10 wildtype short isoform is essential for targeting the long isoform to autophagosomes for degradation, and the mutant short isoform leads to increased PCDHGA10 aggregation and accelerates neurodegeneration. The human mutation identified in Example 1 is in the unique region of the short isoform, which leads to premature stop and truncation of the isoform. Because the long isoform has a constant region that is shared by all other gamma-PCDHs, the short isoform can ensure that protein degradation is specific to PCDHGA10. The size and number of protein aggregates and neuronal survival will be compared under the normal and mutant human PCDHGA10 in neuronal culture of both human and mouse origin. Future studies will generate a CRISPR/Cas9 gene-edited mouse model to demonstrate neurodegeneration in vivo and will examine whether and how PCDHGA10 aggregates change under adverse condition such as oxidative stress or inflammatory responses. Future studies will also determine if the unique region of the short isoform directly interacts with autophagy proteins p62 or LC3.

The findings of this example will establish the timeline of protein aggregation in vestibular ganglia during aging in both humans and mice, providing a comparison of the two species with regards to the window of opportunity for future intervention and whether information gleaned from mouse studies would likely be applicable for clinical cases. In addition, the study will use both in vivo and in vitro approaches to define the role of the PCDHGA10 short isoform in proteostasis during aging and its role in decelerating neurodegeneration in human and mouse vestibular ganglia.

Taken together with data from the literature, the example included herewith suggest that, while BPPV and other protein aggregate diseases (AD, PD, HD, ALS) may be precipitated by different genetic mutations and nongenetic factors, they each may represent a piece of the puzzle, i.e. an event or a component of the cellular pathways underlying proteostasis or lack thereof during aging.

Specific Aim 1: Determine the Approximate Onset Age of PCDHGA10 Protein Aggregation in Human and Mouse Vestibular Ganglia, and its Association with the Proteostasis/Macroautophagy System.

Quantitative fluorescent immunostaining and Western blotting. Human and mouse vestibular cells expressing the various forms of PCDHGA10 will be mapped. In order to do that, antibodies that are specific for human long, short, and mutant short forms, and for mouse long and short forms, must be generated. These antibodies will be used to perform quantitative fluorescent immunostaining of the vestibular ganglia from human postmortem tissues of BPPV and non-BPPV cases, and to quantify sub-cellular distribution of the isoforms at various ages in both humans and mice. Quantitative Western blotting (described below) will be performed to confirm the quantitative differences in total cellular PCDHGA10 protein.

Antibodies for different isoforms will be raised in different species (including, for example, chicken, guinea pigs, and rats) by Novus or Chemicon International for double staining. Initial non-quantitative Western blotting will be used to confirm the antibodies.

Based on an extensive search of the SNP databases for various inbred mouse strains on the websites of the Mouse Genome Informatics by the Jackson Laboratory44 and of the Mouse Genomes Project by the Sanger Institute (The Sanger Institute. Mouse Genomes Project. 2019), it is assumed that the normal mouse does not carry the mutant short form of Pcdhga10. C57Bl/6 (B6) mice will be used because the hypomorphic allele Cdh23753A>G only affects hearing but not vestibular function during aging (Mock et al., 2016, Neurobiol Aging; 43:13-22). In fact, old B6 mice have less vestibular functional loss than CBA/CaJ, a strain with minimal age-related hearing loss (35% vs. 49% vestibular functional loss, respectively, at 24 months vs. 1 month old) (Mock et al., 2016, Neurobiol Aging; 43:13-22; and Mock et al., 2011, J Assoc Res Otolaryngol; 12:173-183). No study has examined vestibular ganglia loss in these mice.

Human postmortem tissues will include samples with normal inner ear function and with BPPV as shown in Table 4. Three female samples with Alzheimer's disease in the >60 age group will also be included for comparison. The control group tallied in the table had normal inner ear function. Those died of cancer are not tallied here even if they had normal inner ear function. Some in the control group had health issues that were not severe (for example, rheumatoid arthritis, scleroderma, and lupus) and were tallied. Most of the young groups tallied here died of various accidents, a few died of anoxia (infants) or drug overdose (young adults). Idiopathic recurrent BPPV is rare in young people.

The samples will be genotyped for presence of the rs113784532 mutation, however, even if absent, studying how PCDHGA10 aggregates in the human vestibular ganglia during aging and how the short isoform modulates this process will still be important information and is a major goal of the proposed project. For comparison, three female ganglia samples with Alzheimer's disease will also be included in the old age group. To co-localize normal (human and mouse tissues) and mutant isoforms (human tissues) of PCDHGA10 with autophagosomes, custom-made antibodies will be triple-stained with LC3 (or p62) (ab192890, Abcam; GP62-C, Progen Biotechnik, respectively) and Casp8/10 (Cat #8592 and 9752, respectively, Cell Signaling Technology). Both LC3 and p62 are specific for autophagosomes and are well-characterized. P62 is a common component found in protein aggregation diseases affecting both the brain and the liver (Zatloukal et al., 2002, Am J Pathol; 160:255-263). These include Lewy bodies in Parkinson's disease, neurofibrillary tangles in Alzheimer's disease, and huntingtin Aggregates (Zatloukal et al., 2002, Am J Pathol; 160:255-263; Kuusisto et al., 2001, Neuroreport; 12:2085-2090; Kuusisto et al., 2002, Neuropathol Appl Neurobiol; 28:228-237; and Nagaoka et al., 2004, J Neurochem; 91:57-68). In the liver, Mallory bodies, hyaline bodies in hepatocellular carcinoma and al antitrypsin aggregates all contain p62. Casp8 and 10 are paralogs, and both are apoptosis-initiator caspases. The human genome has both CASP8 and 10, but the rodent genome does not have Casp10. Because CASP10 is a candidate gene for familial BPPV (see Table 1), human tissues/cells will be stained for CASP10. The size, number, and fluorescent intensity of LC3/p62/PCDHGA10 aggregates and total aggregate area per cell will be quantified using the Analyze Particles tool of ImageJ (n=2 view fields/section×2 sections/tissue×4-6 tissues/age group). Cross sections from both the superior and inferior divisions of the vestibular ganglia will be studied. A power analysis using repeated measures ANOVA showed that this sample size is sufficient to produce 95% statistical power due to the great difference in PCDHGA10 expression between young and aging mice. Because idiopathic recurrent BPPV is rare in young people, this example will focus on the aging group. If there is no gender difference in the young control group, they will be combined as a single age group for comparison, if a larger N is needed.

The above data will be correlated with fluorescent signals of PCDHGA10 long, short, and mutant forms, as well as with neuronal death, at different ages in both sexes. Analysis of the mutant form refers to human tissues only. Otherwise, both human and mouse tissues will be studied for comparison. Neuronal death will be determined by fluorescent staining with Casp8/10 and TUNEL assay for fragmented DNA (TUNEL: Terminal Deoxynucleotidyl-Transferase mediated dUTP Nick End Labelling) (Invitrogen). The Zeiss fluorescent microscope should be sufficient for observing autophagosomes and macroautophagy. If needed, a Zeiss ELYRA PS.1 Super Resolution Microscope will also be used.

To minimize experiment-introduced variations, all samples under comparison will be processed strictly under the same conditions (e.g. identical immunostaining procedures, identical microscope scanning parameters, the same number of fluorescent exposures, and similar background signals). Pre-immune (or nonimmune) sera instead of primary antibodies will be used in some sections as negative controls. Protein levels will be indicated by fluorescent intensity after subtracting tissue backgrounds. P values of measurements in different ages will be obtained using the Student's t-test with Bonferroni correction for multiple comparisons.

The results from qWestern blotting (below) and qRT-PCR (below) will confirm these analyses.

Quantitative Western blotting of mouse vestibular tissues. To confirm the quantitative differences on the Pcdhga10 isoforms in different ages of mouse vestibular ganglia, qWestern blotting will be performed. The isoform protein intensity/total protein content will be used to compare the levels in different ages. Because Western blotting needs many more cells than immunostaining, to conserve human tissues for future mechanistic studies, human tissues will not be included for this experiment or for qRT-PCR below. The findings with mouse tissues should be sufficient to confirm the quantitative fluorescent immunostaining. As mentioned above, the Student's t-test with Bonferroni correction will be used for multiple comparisons to detect statistical significance, and the significant level will be p<0.05.

Quantitative RT-PCR of mouse vestibular tissues. qRT-PCR will be performed to compare the transcript levels of the long and short isoforms in different ages (neonates, 1-2 months old, 9-12 months old, 18-24 months old) of the mouse vestibular ganglia (n=6 in each age and sex group). Although no human samples are included here, the mouse data can be used as reference in term of age differences.

After RNA extraction, genomic DNA will be removed by using the RNase-Free DNase Set (Qiagen) and the RNA Clean and Concentrator columns (Zymo Research Corporation). The Taqman gene expression assays, along with probe and primer sets, will be supplied by ABI (Applied Biosystems). The same amount of cDNA (˜50 ng) will be used in each reaction, and reactions without reverse-transcriptase will be included as well. The cycle number at which the reaction crosses a predetermined cycle threshold (CT) will be identified for each isoform (or each gene), and the expression value of each target gene relative to Actb and Gapdh genes will be determined using the equation 2^{-ΔCT}, where ΔCT=(CT_{Target gene}−CT_{Control}).

Protein aggregation in the vestibular ganglia is expected to start in middle age or older (around 50 years of age or older in humans, 12-15 months or older in mice). Future studies will include (1) using FRET to confirm the direct interactions between PCDHGA10 and macroautophagy proteins; and (2) testing whether and how PCDHGA10 aggregate formation is facilitated by adverse condition such as oxidative stress. The focus of the current work is the comparative aspect of in vivo PCDHGA10 protein aggregates during aging.

### Specific Aim 2: Determining if the PCDHGA10 Wildtype Short Isoform is Essential for Targeting the Long Isoform to Autophagosomes for Degradation, and the Mutant Short Isoform Leads to Increased PCDHGA10 Aggregation and Accelerates Neurodegeneration.

Human neuronal culture will be used to test the effects of the long, short, and mutant forms of PCDHGA10 on protein aggregate formation and neuronal survival. The long and wildtype short isoforms of CDHGA10 will be purchased from Genscript. The mutant short isoform of PCDHGA10 will be generated by subcloning the mutant segment from an affected sample. All constructs will be confirmed by Sanger sequencing. Correct expression will be confirmed by Western blotting.

Human neural progenitor cells (ATCC® ACS-5005™) will be cultured in complete growth media on coverslips coated with CellMatrix Basement Membrane Gel (ATCC® ACS-3035™). The cells will be transfected with:
(i) empty vector pAAV-IRES-hrGFP (or RFP or CFP) as control (Catalog #240075, Stratagene). This vector allows GFP (or RFP or CFP) to be co-transcribed but not co-translated (not fused) with the PCDHGA10 isoform so as not to interfere with the target protein function or sub-cellular localization;
(ii) PCDHGA10 long isoform (GFP);
(iii) wildtype short isoform (RFP);
(iv) mutant short isoform (CFP); (v) long isoform+wildtype short isoform;
(vi) long isoform+mutant short isoform; and
(vii) wildtype+mutant short forms.

In vitro aggresome formation assay. To see if the PCDHGA10 aggregates are caused by insufficient clearance (or inability to be targeted to) by autophagy, cells transfected with PCDHGA10 above will be triple stained with anti-LC3 (or anti-p62) and two isoforms (long/short, long/mutant, or wt short/mutant short) of PCDHGA10. Quantification of aggregates and correlation with levels of PCDHGA10 long, short, and mutant forms, as well as with neuronal death, will be the same as described above in Specific Aim 1 (n=18, consisting of 2 view fields/slide×3 slides/experiment×3 independent experiments per group). A power analysis using repeated measures ANOVA showed that, to achieve α=0.05 (two sided) with a power of 80%, a sample size of 9-15 is needed if the mutant protein has an effect size of 0.8-0.5. In addition to correlation analysis, the averaged percentage of cells with aggregates over total cells, and percentage of apoptotic neurons over total neurons, will be also calculated for each group.

Neuronal survival analysis. To assess the effects of the long, short, and mutant forms of PCDHGA10 on cell viability, the LIVE/DEAD viability/cytotoxicity kit (Thermo Fisher Scientific) will be used. Briefly, at various time points (48, 72, 96 hrs) after transfection, cells will be stained by 2 μM calcein-AM, which labels living cells, and 1 μM ethidium homodimer (EthD-1), which labels dead cells, for 30 min. Hoechst 33258 dye will be used at the same time for total nuclei staining. After washing, cells will be immediately imaged on a Zeiss Axio Observer Z1 inverted fluorescence microscope. Cell death % will be determined by EthD-1-positive vs. Hoechst-positive cells (n=18, consisting of 2 view fields/slide×3 slides/experiment×3 independent experiments per group). The statistical significance of differences among different groups will be determined by one-way ANOVA with Bonferroni correction using SPSS or Excel.

The degree of lysosomal digestion of the expressed long, short, and mutant isoforms of PCDHGA10 will be examined. Whether PCDHGA10 actively participate in autophagosome formation by knocking it down in neuronal culture will also be studied.

### Example 4

### CRISPR/Cas9 Mice

CRISPR/Cas9 mice carrying the identified human frameshift mutation rs113784532 will be generated to quantify longitudinally protein aggregate formation in vivo and the survival rates of vestibular ganglia and identify functional deficits in homozygous and heterozygous mutant mice throughout lifespan.

These PCDHGA10 knockout and knockin mutant mice will be generated using the established CRISPR-Cas9 technology. See, for example, Harms et al., 2014, Curr Protoc Hum Genet; 83:15.7.1-27; Quadros et al., 2015, FEBS Open Bio; 5:191-7; and Miura et al., 2018, Nat Protoc; 13:195-215.

For the knockout model, two guide RNAs will be used for excising out nearly the entire exon 1. Upstream guide: TGCCCTGGGAATCCGCAGCT (SEQ ID NO:11). This guide is designed to cut at the beginning of exon 1 (the guide binding region is conserved in both CBA and C57 mice). Downstream guide: TAAGTAAAATCAGAACTCAC (SEQ ID NO:12). This guide is designed to cut in the intron immediately after the exon to be deleted. An oligonucleotide donor containing an in-frame stop codon is used to join the cleaved ends.

For the knock-in model, one guide RNA and one oligonucleotide donor containing the desired mutation will be used. Guide RNA: TAAGTAAAATCAGAACTCAC (SEQ ID NO:12). This guide is designed to cut near the GTG in the intron to insert a TGA (immediately after GTG).

The guide RNA, Cas9 protein and ssODN donors are commercially procured and injected into CBA/CaJ mouse strain pronuclei.

TABLE 1
Families selected for WES

| | Affected | Unaffected | Total |
|---|---|---|---|
| Family | Individuals | Individuals | sample |
| ID | Sequenced | Sequenced | sequenced |
| FB001 | 2 | 2 | 4 |
| FB002 | 2 | 1 | 3 |
| FB003 | 2 | 1 | 3 |
| FB004 | 3 | 2 | 5 |
| FB005 | 2 | 1 | 3 |
| FB006 | 3 | 0 | 3 |
| FB007 | 3 | 0 | 3 |
| FB008 | 2 | 0 | 2 |
| FB009 | 2 | 0 | 2 |
| FB010 | 3 | 0 | 3 |
| FB011 | 2 | 0 | 2 |
| FB012 | 2 | 0 | 2 |

Whole exome sequencing (WES) and mutation detection (or bioinformatics analysis).

TABLE 2
Variants observed in BPPV versus non-BPPV families.

| | | | | | Mutant allele count (+/−) | | | |
|---|---|---|---|---|---|---|---|---|
| | | | | | BPPV | Non-BPPV | | |
| Gene | Chr | ref | alt | dbSNP | families | families | 1000G/ExAC | NHLBIEA |
| PCDHGA10 | chr5 | | A | rs113784532 | 13/29 | 1/49 | 11/2739 | 502/3428 |
| (P-values) | | | | | | (1.18 × 10⁻⁴) | (1.50 × 10⁻¹⁰⁰) | (4.86 × 10⁻⁴) |
| CASP10 | chr2 | G | A | rs13010627 | 8/29 | 4/49 | 92/2410 | 547/3753 |
| (P-values) | | | | | | (0.05) | (2.54 × 10⁻⁸) | (0.10) |

A total of 42 BPPV families and 50 non-BPPV families were analyzed for rs113784532, and one proband from each family was used for allele frequency calculations. Data are presented as number of families positive (+) for the variant over those negative (−) for it. NHLBI exome sequencing data of European Americans are shown here. Chr, chromosome; EA, European Americans. Data for the control group and for the general population in public genome/exome databases were compared with that of the BPPV group by χ² analysis with Yates' correction, and p values are provided in parentheses.

TABLE 3
Variants and primers used in Sanger sequencing

| | | | | Annealing |
|---|---|---|---|---|
| | | Primer sequence (5′ > 3′) | | Temperature |
| Gene | dbSNP | Forward | Reverse | for PCR (° C.) |
| PCDHGA10 | rs113784532 | AAGAGTCACCTGATCTTCCC | ACACTGGAGTAAAAACCAATCTTTT | 55 |
| CASP10 | rs13010627 | AGGCCCTCATTCCCATTCG | TATACCAGCTGCCTTCCTC | 55 |
| TMEM119 | rs144109867 | CTGCTGATGTTCATCGTCTG | GCTGCCCTTCTCCTCTTC | 60 |
| NOD2 | rs2066847 | TCTTCTTTTCCAGGTTGTCC | GCCTTACCAGACTTCCAG | 55 |
| STARD6 | rs17292725 | AAACTTGCAGATAGATGGAC | TCTTACTGAAGTTTTAACCAC | 55 |
| MYBPC3 | rs3729986 | CTTTGCTCACAGGGTCAAG | ACAGCAGCTCACACTCAC | 55 |
| MPO | rs119468010 | GCATTGACCCCATCCTCC | TGCGCTGCATGTTCAGAG | 60 |
| BAG3 | rs35434411 | CTCCATCCTCTGCCAATG | TCAGTTCGGAATCGCTGC | 60 |
| CP | rs61733458 | GCACCCACAGAAACATTC | AGTTGGACTTACCTGTCTC | 55 |
| LRP2 | rs17848169 | TAGGCAGAGGATTGAACGC | TGGTGCACAGATATGGCTG | 60 |
| LRP2 | rs34291900 | TTCCTTCCAGCTACCAATC | GCGCTCATCTGTATCCAG | 55 |
| SYNE2 | rs12881815 | ACATTGGTGGAAAACACGC | AAAGGCTGACCTGAGCAG | 55 |
| LMNB2 | rs121912497 | CATTTTTGAGCCCTCCCTTG | CTTGGCCTGGTAGGTCTGC | 55 |
| DMD | rs72468681 | TTTTTCTTTCTAGAGGGTG | TTGCTGTTGGCTCTGATG | 55 |
| GPR98 | rs111033530 | TTAGGAGTTCCACCAGCC | GAATTGTTATGTTAGCCTCTTG | 55 |
| CIDEC | rs61742367 | CCAGGCATGTGTCAGTGC | GGTCCTCAAGACTGTAAGC | 55 |
| RYR2 | rs56229512 | TGCTTATTGTTAGTCCTCTG | AAGCTGCAACCTCATACC | 55 |
| ANO10 | rs17409162 | TTTCTTTTAGTTGGCTTTTG | CTTTACTTACCTCCACTG | 52 |

TABLE 4
Human postmortem inner ears already collected and sectioned.

| | | <20 | 21-44 | 45-59 | >60 |
|---|---|---|---|---|---|
| Diagnosis | Gender | N (ages) | N (ages) | N (ages) | N (ages) |
| BPPV | Female | | 3 (25.39,41) | 2 (48,52) | 6 (60,89,75,81,82,83) |
| | Male | | | 2 (48,56)} | 4 (66,71,78,86) |
| Control | Female | 2 (3w, 19) | 6 (21,23,26,27, | 5 (47,51,51,58, | 11 (60,61,68,89,72,72, |
| | | | 29,34) | 59) | 76,77,80,80,82) |
| | Male | 4 (P1,6,15,20) | 11 (21,22,22,23,26, | 9 (47,48,51,52, | 6 (60,62,64,65,66,77) |
| | | | 31,35.37.36,33,43) | 55.55,55,56,58) | |

Sequences
SEQ ID NO: 1 PCDHGA10 polypeptide sequence
SEQ ID NO: 2 PCDHGA10 cDNA
SEQ ID NO: 3 CASP10 polypeptide sequence
SEQ ID NO: 4 CASP10 cDNA
SEQ ID NO: 5 Forward primer, PCDHGA10
SEQ ID NO: 6 Reverse primer, PCDHGA10
SEQ ID NO: 7 Forward primer 1, CASP10
SEQ ID NO: 8 Reverse primer 1, CASP10
SEQ ID NO: 9 Forward primer 2, CASP10
SEQ ID NO: 10 Reverse primer 2, CASP10
SEQ ID NO: 11 Upstream guide (exon 1)
SEQ ID NO: 12 Downstream guide (exon 1)

### EXAMPLE EMBODIMENTS

The inventions defined in the claims. However, below paragraphs provide a non-exhaustive list of non-limiting embodiments. Any one or more of the features of these embodiments may be combined with any one or more features of another example, embodiment, or aspect described herein.
1. A method of diagnosing Benign Paroxysmal Positional Vertigo (BPPV) in a subject, the method comprising determining that at least one allele of the human protocadherin gamma A10 (PCDHGA10) gene in the subject's genome comprises a variation encoding a premature stop codon within intracellular/cytoplasmic domain of the short isoform the human PCDHGA10 polypeptide.
2. A method of diagnosing Benign Paroxysmal Positional Vertigo (BPPV) in a subject, the method comprising genotyping the subject or having the subject genotyped and determining that at least one allele of the human protocadherin gamma A10 (PCDHGA10) gene in the subject's genome comprises a variation encoding a premature stop codon within intracellular/cytoplasmic domain of the short isoform the human PCDHGA10 polypeptide.
3. The method of paragraph 1 or 2, wherein the variation encoding a premature stop codon truncates the intracellular/cytoplasmic domain of the short isoform the human PCDHGA10 polypeptide after about amino acid residue 812.
4. The method of any one of paragraphs 1 to 3, wherein the variation encoding a premature stop codon comprises an insertion mutation.
5. The method of any one of paragraphs 1 to 4, wherein the variation encoding a premature stop codon comprises NM_032090.1:c.2476_2477dup in the protocadherin gamma A10 gene (PCDHGA10).
6. The method of any one of paragraphs 1 to 4, wherein the variation encoding a premature stop codon comprises NM_032090.1:c.2477dup in the protocadherin gamma A10 gene (PCDHGA10).
7. The method of any one of paragraphs 1 to 4, wherein the variation encoding a premature stop codon comprises NM_032090.1:c.2467_2468insA (p.Lys823_Lys824fs) in the protocadherin gamma A10 gene (PCDHGA10).
8. The method of any one of paragraphs 1 to 4, wherein the variation encoding a premature stop codon comprises the SNP allele Rs113784532, rs369101565, rs752029921, or rs750612188.
9. A method of diagnosing Benign Paroxysmal Positional Vertigo (BPPV) in a subject, the method comprising determining that at least one allele of the human Caspase 10 (CASP10) gene in the subject's genome comprises a single nucleotide variation at a position encoding amino acid residue 410.
10. A method of diagnosing Benign Paroxysmal Positional Vertigo (BPPV) in a subject, the method comprising genotyping the subject or having the subject genotyped and determining that at least one allele of the human Caspase 10 (CASP10) gene in the subject's genome comprises a single nucleotide variation at a position encoding amino acid residue 410.
11. The method of paragraph 9 or 10, wherein the variation comprises a valine to isoleucine substitution at position 410.
12. The method of any one of paragraphs 9 to 11, wherein the variation comprises NM_032974.5:c1228G>A.
13. The method of any one of paragraphs 9 to 12, wherein the variation comprises the SNP allele rs13010627.
14. The method of any one of paragraphs 1 to 13, wherein the variation is identified by genomic sequencing.
15. The method of any one of paragraphs 1 to 13, wherein the variation is identified from previously obtained genomic sequencing data.
16. The method of any one of paragraphs 1 to 13, wherein the variation is identified by polymerase chain reaction (PCR).
17. The method of paragraph 16, further comprising sequencing the PCR product.
18. The method of paragraph 16 or 17, wherein the variation comprises a variation encoding a premature stop codon within intracellular/cytoplasmic domain of the short isoform the human PCDHGA10 polypeptide, wherein the forward primer comprises AAGAGTCACCTGATCTTCCC (SEQ ID NO:5), and wherein the reverse primer comprises ACACTGGAGTAAAAACCAATCTTTT (SEQ ID NO:6).
19. The method of paragraph 16 or 17, wherein the variation comprises a variation encoding a premature stop codon within intracellular/cytoplasmic domain of the short isoform the human PCDHGA10 polypeptide, wherein the forward primer comprises ACACTCGAGCTGTGAGAAAAAAGATCC-3′ (SEQ ID NO:7), and wherein the reverse primer comprises ACATCTAGATTTGGGCTCAAGCACAACG (SEQ ID NO:8).
20. The method of paragraph 16 or 17, wherein the variation comprises a variation comprises a single nucleotide variation at position 410 of the human Caspase 10 (CASP10) gene, wherein the forward primer comprises AGGCCCTCATTCCCATTCG (SEQ ID NO:9), and wherein the reverse primer comprises TATACCAGCTGCCTTCCTC (SEQ ID NO:10).
21. The method of any one of paragraphs 1 to 20, further comprising treating the subject with maneuvers to reposition the dislocated otoconia in the utricle, labyrinthectomy, and/or semicircular canal occlusion.
22. The method of any one of paragraphs 1 to 21, further comprising treating the subject with an agent that inhibits the aggregation of neural proteins.
23. A method of treating a subject with Benign Paroxysmal Positional Vertigo (BPPV), the method comprising administering to the subject an inhibitor of the aggregation of neural proteins.
24. The method of any one of paragraphs 1 to 23, wherein the subject has been resistant to treatment with maneuvers to reposition the dislocated otoconia in the utricle.
25. A method of preventing Benign Paroxysmal Positional Vertigo (BPPV) in a subject, the method comprising administering to the subject an inhibitor of the aggregation of neural proteins.
26. The method of any one of paragraphs 22 to 25, wherein the inhibitor of the aggregation of neural proteins inhibits the aggregation of proteins in the vestibular ganglia.
27. A CRISPR/cas9 gene edited mouse comprising at least one allele of the protocadherin gamma A10 (PCDHGA10) gene edited to encode a premature stop codon within intracellular/cytoplasmic domain of the short isoform or comprising at least one allele of the protocadherin gamma A10 (PCDHGA10) gene edited to delete or prevent expression of the short isoform.
28. The CRISPR/cas9 gene edited mouse of paragraph 27, wherein the variation encoding a premature stop codon truncates the intracellular/cytoplasmic domain of the short isoform after about amino acid residues 812.
29. The CRISPR/cas9 gene edited mouse of paragraph 27 or 28, wherein the at least one allele of the protocadherin gamma A10 (PCDHGA10) gene edited to encode a premature stop codon within intracellular/cytoplasmic domain of the short isoform comprises a murine allele or a human allele.
30. A method of identifying an agent that inhibits the aggregation of proteins in neural cells, the method comprising:
administering the agent to the mouse of any one of paragraphs 27 to 29;
assaying protein aggregation in neural cells in the mouse administered the agent;
assaying protein aggregation in neural cells in the mouse to which the agent has not been administered; and
comparing protein aggregation in neural cells in the mouse administered the agent to protein aggregation in neural cells in the mouse to which the agent has not been administered.
31. An antibody that specifically binds to a short isoform of the human protocadherin gamma A10 (PCDHGA10) polypeptide encoded by a variation encoding a premature stop codon within intracellular/cytoplasmic domain of the human PCDHGA10 polypeptide, wherein the antibody does not specifically bind to the wild type short isoform of the human protocadherin gamma A10 (PCDHGA10) polypeptide and does not specifically bind to the wild type long isoform of the human protocadherin gamma A10 (PCDHGA10) polypeptide.
32. The antibody of paragraph 31, wherein the variation encoding a premature stop codon truncates the intracellular/cytoplasmic domain of the short isoform the human PCDHGA10 polypeptide after about amino acid residues 812.
33. The antibody of paragraph 31 or 32, wherein the variation encoding a premature stop codon comprises an insertion mutation.
34. The antibody of any one of paragraphs 31 to 33, wherein the variation encoding a premature stop codon comprises:
NM_032090.1:c.2476_2477dup in the protocadherin gamma A10 gene (PCDHGA10);
NM_032090.1:c.2477dup in the protocadherin gamma A10 gene (PCDHGA10);
NM_032090.1:c.2467_2468insA (p.Lys823_Lys824fs) in the protocadherin gamma A10 gene (PCDHGA10); or
the SNP allele Rs113784532, rs369101565, rs752029921, or rs750612188.
35. A method of monitoring protein aggregation in neural cells, the method comprising contacting the neural cells with an antibody of any one of paragraphs 31 to 34.
36. A method of identifying an agent that inhibits the aggregation of proteins in the vestibular ganglia, the method comprising:
culturing in vitro neural cells expressing a human protocadherin gamma A10 (PCDHGA10) polypeptide encoded by a PCDHGA10 gene comprising a premature stop codon within intracellular/cytoplasmic domain of the short isoform the human PCDHGA10 polypeptide;
contacting the cells with the agent;
assaying for protein aggregation in the cells.
37. The method of paragraph 36, wherein the premature stop codon truncates the intracellular/cytoplasmic domain of the short isoform the human PCDHGA10 polypeptide after about amino acid residues 812.
38. The method of paragraph 36 or 37, wherein the premature stop codon comprises an insertion mutation.
39. The method of any one of paragraphs 36 to 38, wherein the premature stop codon comprises:
NM_032090.1:c.2476_2477dup in the protocadherin gamma A10 gene (PCDHGA10);
NM_032090.1:c.2477dup in the protocadherin gamma A10 gene (PCDHGA10);
NM_032090.1:c.2467_2468insA (p.Lys823_Lys824fs) in the protocadherin gamma A10 gene (PCDHGA10); or the SNP allele Rs113784532, rs369101565, rs752029921, or rs750612188.
40. The method of any one of paragraphs 36 to 39, wherein protein aggregation in the cells is assayed with an antibody of any one of paragraphs 31 to 34.

The complete disclosure of all patents, patent applications, and publications, and electronically available material (including, for instance, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

What is claimed is:

1. A method of diagnosing Benign Paroxysmal Positional Vertigo (BPPV) in a subject, the method comprising determining that at least one allele of the human protocadherin gamma A10 (PCDHGA10) gene in the subject's genome comprises a variation encoding a premature stop codon within intracellular/cytoplasmic domain of the short isoform the human PCDHGA10 polypeptide.

2. The method of claim 1, wherein the determining comprises genotyping the subject or having the subject genotyped.

3. The method of claim 1, wherein the variation encoding a premature stop codon truncates the intracellular/cytoplasmic domain of the short isoform the human PCDHGA10 polypeptide after about amino acid residue 812.

4. The method of claim 1, wherein the variation encoding a premature stop codon comprises an insertion mutation.

5. The method of claim 1, wherein the variation encoding a premature stop codon comprises NM_032090.1:c.2476_2477dup in the protocadherin gamma A10 gene (PCDHGA10).

6. The method of claim 1, wherein the variation encoding a premature stop codon comprises NM_032090.1:c.2477dup in the protocadherin gamma A10 gene (PCDHGA10).

7. The method of claim 1, wherein the variation encoding a premature stop codon comprises NM_032090.1:c.2467_2468insA (p.Lys823_Lys824fs) in the protocadherin gamma A10 gene (PCDHGA10).

8. The method of claim 1, wherein the variation encoding a premature stop codon comprises the SNP allele Rs113784532, rs369101565, rs752029921, or rs750612188.

9. A method of diagnosing Benign Paroxysmal Positional Vertigo (BPPV) in a subject, the method comprising determining that at least one allele of the human Caspase 10 (CASP10) gene in the subject's genome comprises a single nucleotide variation at a position encoding amino acid residue 410.

10. The method of claim 9, wherein the determining comprises genotyping the subject or having the subject genotyped.

11. The method of claim 9, wherein the variation comprises a valine to isoleucine substitution at position 410.

12. The method of claim 9, wherein the variation comprises NM_032974.5:c1228G>A.

13. The method of claim 9, wherein the variation comprises the SNP allele rs13010627.

14. The method of claim 1, further comprising treating the subject by administering to the subject an inhibitor of the aggregation of neural proteins.

15. The method of claim 14, wherein the inhibitor of the aggregation of neural proteins inhibits the aggregation of proteins in the vestibular ganglia.

16. An antibody that specifically binds to a short isoform of the human protocadherin gamma A10 (PCDHGA10) polypeptide encoded by a variation encoding a premature stop codon within intracellular/cytoplasmic domain of the human PCDHGA10 polypeptide, wherein the antibody does not specifically bind to the wild type short isoform of the human protocadherin gamma A10 (PCDHGA10) polypeptide and does not specifically bind to the wild type long isoform of the human protocadherin gamma A10 (PCDHGA10) polypeptide.

17. The antibody of claim 16, wherein the variation encoding a premature stop codon truncates the intracellular/cytoplasmic domain of the short isoform the human PCDHGA10 polypeptide after about amino acid residues 812.

18. The antibody of claim 16, wherein the variation encoding a premature stop codon comprises an insertion mutation.

19. The antibody of claim 16, wherein the variation encoding a premature stop codon comprises:
NM_032090.1:c.2476_2477dup in the protocadherin gamma A10 gene (PCDHGA10);
NM_032090.1:c.2477dup in the protocadherin gamma A10 gene (PCDHGA10);
NM_032090.1:c.2467_2468insA (p.Lys823_Lys824fs) in the protocadherin gamma A10 gene (PCDHGA10); or
the SNP allele Rs113784532, rs369101565, rs752029921, or rs750612188.
